SEQUENCE LISTING (50 sequences)

SEQ ID NO: 1 - DNA, 2015 bp, Oreochromis niloticus
  source 1..2015: mol_type = genomic DNA
tgctcagaccttcacatgcaaaatagagctgcgtggtgcttgaactctttgttcagctatgcagtagtgaagaggattaactcaagccaagcgccagttttggcatttcatccatcccttggctgacggagagagactaccaaaagctacctctttgcctctcttcatctaaatctcctctttactcccttcctgttctctttttctccttctgtccttttccccactctttttcaattatgactctgttatcagaagatcagtctgtgaggccccaaccctgccgggtctctgagcctggcaaacacatcagcacttctctgcaccagtactcaacagactgcaatggcagcaatgtggactatgtagatagtgtggaagactctgtctttggagctgtggagcctcacaggcgctctcgggggcgccttattctccacggcctggtcatgtttggaagggaattctgctatgccgtggaggcagcttttgtcacgccggtgctcctgagtgtgggcctgccccagagtctgtacagcttggtgtggttgatcagccccatcctgggcttcctgctccagcccatcatcggttcagccagcgactattgccgctcgccgtggggccggcggaggccttacatcctggcactgggcatcctcatgcttgtgggaatcaccctgttccttaatggagatgctgtcatctcagcactcgtcagagataggtcgctgaagagcatatgggccatcgtggtggtgatgtttggagtcgtgctgtttgactttgctgccgacttcatcgatggacccattaaggcctacctgtttgatgtgtgttcacatgaagacaaggaaagaggcttgcactaccatgctctcctcacaggcctgggcggagcatgtggttacctggtgggagccatggactggggtcactccgtgttgggccgtctccttggatccgagtaccaggtcatctatttcttctcagcgctgacctggggcatcttcctcactgtgcacctcttcagtattccagagcaacctctggacaaggcaccatctccgtctaattcaccatccagccctcttcgcctactgagctctcatagcaatagttatggagcacttaaggagcctgtctctcctgtagtctccgcatctgtcacagatctcaggcccagatccttctcagccctgggagaagccaattcagtgacctcaagcgtcaaacagccaaacaaagaggaccagaagaagatgacgttcaggttattgataaaagctatcgtcaatatgccaagtcactaccgctgcctgtgtgtcagccacctgctgggatggacagcgttcctctgcaatatgcttttctttaccgatttcatgggacagatcgtgtataaggggaatccgtacgccgatcacaactctaccgcctatgccacatatgagaggggagtagaagttggctgctggggattgtgcattaatgcagtgtcctctgctctctactcatatgtgcagcgcttcctcctctcatacatcggactgaagggattatattttatgggctaccttgtgtttggattgggcaccagcctgattggccttttccccaatatcatagcaacgctcgtcctctgcagtgtgtttggtgttatgtcaagcacactctacaccatcccattcaacctgatagcggagtatcaacgtgaagaggagcaacaactgaagctgcacggaagcaataaaagtcctcgaggctcgggaatggactgtgccgccctcacttgcatggtccagctggctcagatcatcgtgggcgtggggctcggtgctctggtcaacgcggcaggaagtgtgatcgtggtggtcctctcggcttcgaccatgtctctgctcggctgcatcttcattgtcctcttcatcagatacgtggaatgaatgattctgctcaaatgcaaattttaatgatattgttttaataaacaatgattgctcattatatcatca

SEQ ID NO: 2 - protein, 568 aa, Oreochromis niloticus
MTLLSEDQSVRPQPCRVSEPGKHISTSLHQYSTDCNGSNVDYVDSVEDSVFGAVEPHRRSRGRLILHGLVMFGREFCYAVEAAFVTPVLLSVGLPQSLYSLVWLISPILGFLLQPIIGSASDYCRSPWGRRRPYILALGILMLVGITLFLNGDAVISALVRDRSLKSIWAIVVVMFGVVLFDFAADFIDGPIKAYLFDVCSHEDKERGLHYHALLTGLGGACGYLVGAMDWGHSVLGRLLGSEYQVIYFFSALTWGIFLTVHLFSIPEQPLDKAPSPSNSPSSPLRLLSSHSNSYGALKEPVSPVVSASVTDLRPRSFSALGEANSVTSSVKQPNKEDQKKMTFRLLIKAIVNMPSHYRCLCVSHLLGWTAFLCNMLFFTDFMGQIVYKGNPYADHNSTAYATYERGVEVGCWGLCINAVSSALYSYVQRFLLSYIGLKGLYFMGYLVFGLGTSLIGLFPNIIATLVLCSVFGVMSSTLYTIPFNLIAEYQREEEQQLKLHGSNKSPRGSGMDCAALTCMVQLAQIIVGVGLGALVNAAGSVIVVVLSASTMSLLGCIFIVLFIRYVE

SEQ ID NO: 3 - DNA, 20 bp, synthetic construct
  misc_feature 1..20: note = Single strand DNA oligonucleotide
  source 1..20: mol_type = other DNA
ctggcaaacacatcagcact

SEQ ID NO: 4 - DNA, 20 bp, synthetic construct
  misc_feature 1..20: note = Single strand DNA oligonucleotide
  source 1..20: mol_type = other DNA
ttcctcaccctcacgttttc

SEQ ID NO: 5 - DNA, 22 bp, synthetic construct
  misc_feature 1..22: note = Single strand DNA oligonucleotide
  source 1..22: mol_type = other DNA
tgtacagcttggtgtggttgat

SEQ ID NO: 6 - DNA, 21 bp, synthetic construct
  misc_feature 1..21: note = Single strand DNA oligonucleotide
  source 1..21: mol_type = other DNA
gtgccaggatgtaaggcctcc

SEQ ID NO: 7 - DNA, 63 bp, synthetic construct
  misc_feature 1..63: note = Single strand DNA oligonucleotide
  source 1..63: mol_type = other DNA
taatacgactcactatagggggaattctgctatgccgtgggttttagagctagaaatagcaag

SEQ ID NO: 8 - DNA, 80 bp, synthetic construct
  misc_feature 1..80: note = Single strand DNA oligonucleotide
  source 1..80: mol_type = other DNA
aaaagcaccgactcggtgccactttttcaagttgataacggactagccttattttaacttgctatttctagctctaaaac

SEQ ID NO: 9 - DNA, 22 bp, synthetic construct
  misc_feature 1..22: note = gRNA target seq
  source 1..22: mol_type = other DNA
agggaattctgctatgccgtgg

SEQ ID NO: 10 - DNA, 22 bp, synthetic construct
  misc_feature 1..22: note = gRNA target seq
  source 1..22: mol_type = other DNA
tcgctggctgaaccgatgatgg

SEQ ID NO: 11 - DNA, 22 bp, synthetic construct
  misc_feature 1..22: note = gRNA target seq
  source 1..22: mol_type = other DNA
ttgccgctcgccgtggggccgg

SEQ ID NO: 12 - DNA, 20 bp, synthetic construct
  misc_feature 1..20: note = gRNA target seq
  source 1..20: mol_type = other DNA
ggaattctgctatgccgtgg

SEQ ID NO: 13 - RNA, 29 nt, synthetic construct
  misc_feature 1..29: note = gRNA nucleic acid sequence
  source 1..29: mol_type = other RNA
agggaattctgctatgccgatgctgtttg

SEQ ID NO: 14 - RNA, 29 nt, synthetic construct
  misc_feature 1..29: note = gRNA nucleic acid sequence
  source 1..29: mol_type = other RNA
tcgctggctgaaccgatgaatgctgtttg

SEQ ID NO: 15 - RNA, 29 nt, synthetic construct
  misc_feature 1..29: note = gRNA nucleic acid sequence
  source 1..29: mol_type = other RNA
ttgccgctcgccgtggggcatgctgtttg

SEQ ID NO: 16 - RNA, 100 nt, synthetic construct
  misc_feature 1..100: note = sgRNA nucleic acid sequence
  source 1..100: mol_type = other RNA
ggaattctgctatgccgtgggttttagagctagaaatagcaagttaaaataaggctagtccgttatcaacttgaaaaagtggcaccgagtcggtgctttt

SEQ ID NO: 17 - DNA, 451 bp, synthetic construct
  misc_feature 1..451: note = sequencing of gDNA product
  source 1..451: mol_type = other DNA
ctggcaaacacatcagcacttctctgcaccagtactcaacagactgcaatggcagcaatgtggactatgtagatagtgtggaagactctgtctttggagctgtggagcctcacaggcgctctcgggggcgccttattctccacggcctggtcatgtttggaagggaattctgctatgccgtggaggcagcttttgtcacgccggtgctcctgagtgtgggcctgccccagagtctgtacagcttggtgtggttgatcagccccatcctgggcttcctgcttcagcccatcatcggttcagccagcgactattgccgctcgccgtggggccggcggaggccttacatcctggcactgggcatcctcatgcttgtgggaatcaccctgttccttaatggagatgctgtcatctcaggtgagggatgagtgaagaaaacgtgagggtgaggaaa

SEQ ID NO: 18 - DNA, 431 bp, synthetic construct
  misc_feature 1..431: note = sequencing of gDNA product
  source 1..431: mol_type = other DNA
ttcctcaccctcacgttttcttcactcatccctcacctgagatgacagcatctccattaaggaacagggtgattcccacaagcatgaggatgcccagtgccaggatgtaaggcctccgccggcggcaatagtcgctggctgaaccgatgatgggctggagcaggaagcccaggatggggctgatcaaccacaccaagctgtacagactctggggcaggcccacactcaggagcaccggcgtgacaaaagctgcctccacggaattcccttccaaacatgaccaggccgtggagaataaggcgcccccgagagcgcctgtgaggctccacagctccaaagacagagtcttccacactatctacatagtccacattgctgccattgcagtctgttgagtactggtgcagagaagtgctgatgtgtttgccaga

SEQ ID NO: 19 - DNA, 456 bp, synthetic construct
  misc_feature 1..456: note = sequencing of gDNA product
  source 1..456: mol_type = other DNA
ttcctcaccctcacgttttcttcactcatccctcacctgagatgacagcgtctccattaaggaacagggtgattcccacaagcatgaggatgcccagtgccaggatgtaaggcctccgccggcgagcggcaatagtcgctggctgaaccgatgggctggagcaggaagcccaggatggggctgatcaaccacaccaagctgtacagactctggggcagggtctgtaccctctggggcaggcccacactcaggagcaccggcgtgacaaaagctgcctccatagcagaattcccttccaaacatgaccaggccgtggagaataaggcgcccccgagagcgcctgtgaggccccacagctccaaagacagagtcttccacactatctacatagtccacattgctgccattgcagtctgttgagtactggtgcagagaagtgctgatgtgtttgccaga

SEQ ID NO: 20 - DNA, 448 bp, synthetic construct
  misc_feature 1..448: note = sequencing of gDNA product
  source 1..448: mol_type = other DNA
ctggcaaacacatcagcacttctctgcaccagtactcaacagactgcaatggcagcaatgtggactatgtagatagtgtggaagactctgtctttggagctgtggagcctcacaggcgctctcgggggcgccttattctccacggcctggtcatgtttggaagggaattctgctatggaggcagcttttgtcacgccggtgctcctgagtgtgggcctgccccagagtctgtacagcttggtgtggttgatcagccccatcctgggcttcctgctccagcccatcatcggttcagccagcgactattgccgctcgccgtggctcgccggcggaggccttacatcctggcactgggcatcctcatgcttgtgggaatcaccctgttccttaatggagatgctgtcatctcaggtgagggatgagtgaagaaaacgtgagggtgaggaaa

SEQ ID NO: 21 - DNA, 445 bp, synthetic construct
  misc_feature 1..445: note = sequencing of gDNA product
  source 1..445: mol_type = other DNA
ctggcaaacacatcagcacttctctgcaccagtactcaacagactgcaatggcagcaatgtggactatgtagatagtgtggaagactctgtctttggagctgtggagcctcacaggcgctctcgggggcgccttattctccacggcctggtcatgtttggaagggaattctgccgtggaggcagcttttgtcacgccggtgctcctgagtgtgggcctgccccagagtctgtacagcttggtgtggttgatcagccccatcctgggcttcctgctccagcccatcggctcagccagcgactattgccgctcgccgtggctcgccggcggaggccttacatcctggcactgggcaccctcatgcttgtgggaatcaccctgttccttaatggagatgctgtcatctcaggtgagggatgagtgaagaaaacgtgagggtgaggaaa

SEQ ID NO: 22 - DNA, 121 bp, synthetic construct
  misc_feature 1..121: note = sequencing of gDNA product
  source 1..121: mol_type = other DNA
tgtacagcttggtgtggttgatcagccccatcctgggcttcctgctccagcccatcatcggttcagccagcgactattgccgctcgccgtggggccggcggaggccttacatcctggcaca

SEQ ID NO: 23 - DNA, 113 bp, synthetic construct
  misc_feature 1..113: note = sequencing of gDNA product
  source 1..113: mol_type = other DNA
tgtacagcttggtgtggttgatcagccccatcctgggcttcctgctccagcccatcagtcggttcagccagcgactattgccgcccgccgcggaggccttacatcctggcaca

SEQ ID NO: 24 - DNA, 109 bp, synthetic construct
  misc_feature 1..109: note = sequencing of gDNA product
  source 1..109: mol_type = other DNA
tgtacagcttggtgtggttgatcagccccatcctgggcttcctgctccagcccatcggttcatccagccactatcgacggtcgccccgcacgcattacttcctgcctga

SEQ ID NO: 25 - DNA, 112 bp, synthetic construct
  misc_feature 1..112: note = sequencing of gDNA product
  source 1..112: mol_type = other DNA
tgtacagcttggtgtggttgatcagccccatcctgggcttcctgctccagcccatcatcggttcagccagcgactattgccgctcgccgcggaggccttacatcctggcaca

SEQ ID NO: 26 - DNA, 64 bp, synthetic construct
  misc_feature 1..64: note = sequencing of gDNA product
  source 1..64: mol_type = other DNA
ttgtacagcttggtgtggttgatcagccccatcctgggccggcggaggccttacatcctggcac

SEQ ID NO: 27 - DNA, 114 bp, synthetic construct
  misc_feature 1..114: note = sequencing of gDNA product
  source 1..114: mol_type = other DNA
tgtacagcttggtgtggttgatcagccccatcctgggcttcctgctccagcccatcagtcggttcagccagcgactattgccgctcgccggcggaggccttacatcctggcaca

SEQ ID NO: 28 - DNA, 111 bp, synthetic construct
  misc_feature 1..111: note = sequencing of gDNA product
  source 1..111: mol_type = other DNA
tgtacagcttggtgtggttgatcagccccatcctgggcttcctgctccagcccatcggttcagccagcgactattgccgctcgccgtgcggaggccttacatcctggcaca

SEQ ID NO: 29 - DNA, 111 bp, synthetic construct
  misc_feature 1..111: note = sequencing of gDNA product
  source 1..111: mol_type = other DNA
ttgtacagcttggtgtggttgatcagccccatcctgggcttcctgctccagcccatcggttcagccagcgactattgccgctcgccgtgcggaggccttacatcctggcac

SEQ ID NO: 30 - DNA, 115 bp, synthetic construct
  misc_feature 1..115: note = sequencing of gDNA product
  source 1..115: mol_type = other DNA
tgtacagcttggtgtggttgatcagccccatcctgggcttcctgctccagcccatcatcggttcagccagcgactattgccgctcgccgtggcggaggccttacatcctggcaca

SEQ ID NO: 31 - DNA, 112 bp, synthetic construct
  misc_feature 1..112: note = sequencing of gDNA product
  source 1..112: mol_type = other DNA
tgtacagcttggtgtggttgatcagccccatcctgggcttcctgctccagcccatcatcggttcagccagcgactattgccctcgccggcggaggccttacatcctggcaca

SEQ ID NO: 32 - DNA, 117 bp, synthetic construct
  misc_feature 1..117: note = sequencing of gDNA product
  source 1..117: mol_type = other DNA
ttgtacagcttggtgtggttgatcagccccatcctgggcttcctgctccagcccatcagcccatcggttcagccagcgactattgccgctcgccgcggaggccttacatcctggcac

SEQ ID NO: 33 - DNA, 64 bp, synthetic construct
  misc_feature 1..64: note = sequencing of gDNA product
  source 1..64: mol_type = other DNA
tgtacagcttggtgtggttgatcagccccatcctgggccggcggaggccttacatcctggcaca

SEQ ID NO: 34 - DNA, 76 bp, synthetic construct
  misc_feature 1..76: note = sequencing of gDNA product
  source 1..76: mol_type = other DNA
agccccatcctgggcttcctgctccagcccatcagccggttcagccagcgactattgccctcgccggcggaggcct

SEQ ID NO: 35 - DNA, 75 bp, synthetic construct
  misc_feature 1..75: note = sequencing of gDNA product
  source 1..75: mol_type = other DNA
agccccatcctgggcttcctgctccagcccatcatcggttcagccagcggctattgccctcgccggcggaggcct

SEQ ID NO: 36 - DNA, 111 bp, synthetic construct; identical to SEQ ID NO: 28
  misc_feature 1..111: note = sequencing of gDNA product
  source 1..111: mol_type = other DNA

SEQ ID NO: 37 - DNA, 116 bp, synthetic construct
  misc_feature 1..116: note = sequencing of gDNA product
  source 1..116: mol_type = other DNA
tgtacagcttggtgtggttgatcagccccatcctgggcttcctgctccagcccatcagttcagccagcgactattgccgctcgccagggccggcggaggccttacatcctggcaca

SEQ ID NO: 38 - DNA, 132 bp, synthetic construct
  misc_feature 1..132: note = sequencing of gDNA product
  source 1..132: mol_type = other DNA
ttgtacagcttggtgtggttgatcagccccatcctgggcttcctgctccagcccatcaatagtcgctggctgagcccatcggttcagccagcgactattgccgctcgccgcggaggccttacatcctggcac

SEQ ID NO: 39 - DNA, 115 bp, synthetic construct; identical to SEQ ID NO: 30
  misc_feature 1..115: note = sequencing of gDNA product
  source 1..115: mol_type = other DNA

SEQ ID NO: 40 - DNA, 112 bp, synthetic construct; identical to SEQ ID NO: 31
  misc_feature 1..112: note = sequencing of gDNA product
  source 1..112: mol_type = other DNA

SEQ ID NO: 41 - DNA, 117 bp, synthetic construct; identical to SEQ ID NO: 32
  misc_feature 1..117: note = sequencing of gDNA product
  source 1..117: mol_type = other DNA

SEQ ID NO: 42 - DNA, 76 bp, synthetic construct
  misc_feature 1..76: note = sequencing of gDNA product
  source 1..76: mol_type = other DNA
agccccatcctgggcttcctgctccagcccatcaatcggttcagccagcgactattgccctcgccggcggaggcct

SEQ ID NO: 43 - DNA, 64 bp, synthetic construct; identical to SEQ ID NO: 26
  misc_feature 1..64: note = sequencing of gDNA product
  source 1..64: mol_type = other DNA

SEQ ID NO: 44 - DNA, 76 bp, synthetic construct; identical to SEQ ID NO: 34
  misc_feature 1..76: note = sequencing of gDNA product
  source 1..76: mol_type = other DNA

SEQ ID NO: 45 - DNA, 75 bp, synthetic construct; identical to SEQ ID NO: 35
  misc_feature 1..75: note = sequencing of gDNA product
  source 1..75: mol_type = other DNA

SEQ ID NO: 46 - DNA, 472 bp, synthetic construct
  misc_feature 1..472: note = genomic slc45a2-exon1 from genomic database (chrLG7 (reverse strand): 16157420- 16156969
  source 1..472: mol_type = other DNA
atgactctgttatcagaagatcagtctgtgaggccccaaccctgccgggtctctgagcctggcaaacacatcagcacttctctgcaccagtactcaacagactgcaatggcagcaatgtggactatgtagatagtgtggaagactctgtctttggagctgtggagcctcacaggcgctctcgggggcgccttattctccacggcctggtcatgtttggaagggaattctgctatgccgtggaggcagcttttgtcacgccggtgctcctgagtgtgggcctgccccagagtctgtacagcttggtgtggttgatcagccccatcctgggcttcctgctccagcccatcatcggttcagccagcgactattgccgctcgccgtggggccggcggaggccttacatcctggcactgggcatcctcatgcttgtgggaatcaccctgttccttaatggagatgctgtcatctcag

SEQ ID NO: 47 - DNA, 451 bp, synthetic construct
  misc_feature 1..451: note = Nile tilapia broodstock allel 1
  source 1..451: mol_type = other DNA
ctggcaaacacatcagcacttctctgcaccagtactcaacagactgcaatggcagcaatgtggactatgtagatagtgtggaagactccgtctttggagctgtggagcctcacaggcgctctcgggggcgccttattctccacggcctggtcatgtttggaagggaattctgctatgccgtggaggcagcttttgtcacgccggtgctcctgagtgtgggcctgccccagagtctgtacagcttggtgtggttgatcagccccatcctgggcttcctgctccagcccatcatcggttcagccagcgactattgccgctcgccgtggggccggcggaggccttacatcctggcactgggcatcctcatgcttgtgggaatcaccctgttccttaatggagatgctgtcatctcaggtgagggatgagtgaagaaaacgtgagggtgaggaaa

SEQ ID NO: 48 - DNA, 451 bp, synthetic construct; identical to SEQ ID NO: 17
  misc_feature 1..451: note = Nile tilapia broodstock allel 2
  source 1..451: mol_type = other DNA

SEQ ID NO: 49 - DNA, 55 bp, synthetic construct
  misc_feature 1..55: note = Single strand DNA oligonucleotide
  source 1..55: mol_type = other DNA
acactctttccctacacgacgctcttccgatcttgtacagcttggtgtggttgat

SEQ ID NO: 50 - DNA, 55 bp, synthetic construct
  misc_feature 1..55: note = Single strand DNA oligonucleotide
  source 1..55: mol_type = other DNA
gtgactggagttcagacgtgtgctcttccgatctgtgccaggatgtaaggcctcc

(19) United States
(12) Patent Application Publication
(10) Pub. No.: US 2023/0413790 A1
(43) Pub. Date: Dec. 28, 2023

(54) GENETICALLY EDITED ALBINO-RED GERMLINES OF TILAPIA FISH

(71) Applicant: The State of Israel, Ministry of Agriculture & Rural Development, Agricultural Research Organization, Rishon-LeZion (IL)

(72) Inventors: Jakob BIRAN, Petah Tikva (IL); Avner CNAANI, Beit Hashmonai (IL); Adi SEGEV, Rehovot (IL); Tatiana SLOSMAN, Lod (IL)

(73) Assignee: The State of Israel, Ministry of Agriculture & Rural Development, Agricultural Research Organization, Rishon-LeZion (IL)

(21) Appl. No.: 18/197,151

(22) Filed: May 15, 2023

Related U.S. Application Data
(63) Continuation of application No. PCT/IL2021/051354, filed on Nov. 15, 2021.
(60) Provisional application No. 63/113,892, filed on Nov. 15, 2020.

(51) Int. Cl. A01K 67/027

(52) U.S. Cl. CPC A01K 67/0275; A01K 2227/40; A01K 2217/075

(57) ABSTRACT

A fish of a tilapia genus comprising a loss-of-function mutation in a slc45a2 gene, wherein the mutation is in a homozygous form, and wherein the loss-of-function mutation results in an albino-red phenotype of the fish is disclosed. Methods of generating the fish are also disclosed.

### RELATED APPLICATIONS

This application is a Continuation (CON) Patent Application of PCT Application No. PCT/IL2021/051354 filed on Nov. 15, 2021, which claims the benefit of priority of under 35 USC § 119(e) of U.S. Provisional Patent Application No. 63/113,892 filed on Nov. 15, 2020. The contents of the above applications are all incorporated by reference as if fully set forth herein in their entirety.

### SEQUENCE LISTING STATEMENT

The XML file, entitled 96610ReplacementSequenceListing.xml, created on Aug. 27, 2023, comprises 70,328 bytes.

### FIELD AND BACKGROUND OF THE INVENTION

The present invention, in some embodiments thereof, relates to albino-red tilapia strains devoid of grey/black pigmentation and, more particularly, but not exclusively, to generation of same by gene editing.

Tilapias are the second most aquacultured fish group in the world, with a global production of roughly 5.5 million tons per year, most of which comprise production of Nile tilapia (Oreochromis niloticus). In recent years, red tilapias, which are usually different hybrid strains of Nile tilapia crossed with Mozambique, blue, and Zanzibar tilapias (O. mossambicus, O. aureus, O. hornorum), are gaining worldwide popularity and their prices increase accordingly. The market price of red tilapia in the Philippines is double than the wild-type (grey) tilapia, and for similar reasons, 85% of the tilapias grown in Malaysia are red tilapia strains. It has been suggested that the red coloration is due to faulty melanophores development, however, this results in a non-stable red phenotype with black or dark-red color blotches which reduce the fish market value. An alternative approach of phenotypic selection for red coloration has resulted in a significant loss of genetic variability due to a founders effect. Moreover, most current red tilapia strains display reduced growth compared to other commercially used Nile tilapia strains.

Previous studies have focused on genes related to body colors in fish. It is evident that pigment synthesis is a complex process that involves multiple genes including, e.g. tyrosinase, tyrosinase-related protein 1, dopachrome tautomerase, silver and others. Furthermore, fish-specific genomic duplications resulted in multiple genomic copies for most of these genes. In teleostei genomes, most genes involved in regulation of melanin synthesis are duplicated, with some exceptions, including e.g. slc45a2, oculocutaneous albinism type 2 (oca2) and solute carrier family 24 member 5 (slc24a5), as they are encoded by a single gene [Braasch, I. et al., BMC Evolutionary Biology (2007) 7: 74].

Solute carrier family 45 member 2 (SLC45A2), also known as membrane-associated transporter protein (MATP), absent in melanoma-1 (Aim-1), oculocutaneous albinism type 4 (OCA4), B gene and as albino (alb), is an evolutionarily conserved key mediator of melanin biosynthesis. SLC45A2 shares high similarity with sucrose transporter proteins in animals and plants, however it is likely to affect tyrosinase activity through regulation of melanosomal pH.

It was previously suggested that the red phenotype in tilapia results from dermis blood irrigation in perturbed melanophore development fish [Hilsdorf A. W. S. et al., Pigment cell research/sponsored by the European Society for Pigment Cell Research and the International Pigment Cell Society (2002) 15: 57-61]. Nonetheless, red to albino-like pink tilapia strains display black eye pigmentation and is usually accompanied with variable rate and pattern of black blotching [Lago, A. et al., Journal of Applied Genetics (2019) 60: 393-400]. Contrastingly, naturally occurring loss-of-function mutations in slc45a2 gene resulted in complete and heritable albinism in medaka [Fukamachi, S. et al., Nature Genetics (2001) 28: 381-385] and zebrafish [Dooley, C. M. et al., Pigment Cell & Melanoma Research (2013) 26: 205-217], and was transiently phenocopied in Atlantic salmon (Salmo salar) [Edvardsen, R. B. et al., PLoS ONE (2014) 9] and marine medaka (Oryzias melastigma) [Jeong, C. B. et al., Marine pollution bulletin (2020) 154, 111038]. Since red to albino-like tilapia strains display black eye pigmentation and/or black blotching it was suggested that it would be less likely that the available strains of red tilapia have resulted from a naturally occurring null mutation in the slc45a2 gene.

In known strains of red tilapia, it has been suggested that the red phenotype is controlled by different genes, these being associated with at least three different linkage groups—chrLG3, chrLG5 and chrLG15 [Li et al., Marine Biotechnology (2019) 21:384-395], and with different modes of inheritance—from dominant red allele, through recessive red allele, to heterozygous red [Li et al. (2019) supra]. Moreover, the distribution and intensity of black blotches in these red tilapia strains are controlled by separate genes than the red-determining ones.

Several methods allowing reverse genetics in fish were developed in the recent years. These include the Zinc-Finger nucleases, TALEN and CRISPR-Cas9 methods. Beyond the high scientific potential and interest held by these technologies, they also possess great potential for agricultural related applications, such as perturbation of genes with a negative agricultural-commercial value. Currently, the CRISPR-Cas9 method is considered as the most efficient, due to its relative technical-simplicity and because of its high mutagenesis rates in vivo. Furthermore, it was recently demonstrated that the high effectiveness CRISPR-Cas9 system can allow the characterization of phenotypes resulting from loss of function mutations already in the injected fish (F₀). Indeed, microinjection of slc45a2—or tyr-specific gRNA with Cas9 mRNA resulted in somatic deletions of tyr gene and reduced melanin formation in the skin of salmon and lamprey larvae [Edvardsen, R. B. et al., (2014), supra].

### SUMMARY OF THE INVENTION

According to an aspect of some embodiments of the present invention there is provided a fish of a tilapia genus comprising a loss-of-function mutation in a slc45a2 gene, wherein the mutation is in a homozygous form, and wherein the loss-of-function mutation results in an albino-red phenotype of the fish.

According to an aspect of some embodiments of the present invention there is provided a method of generating the fish of some embodiments of the invention, the method comprising: (a) introducing into a zygote of the fish of the tilapia genus a DNA editing agent conferring a loss-of-function mutation in the slc45a2 gene; (b) allowing the zygote of step (a) to develop into a fish, thereby generating the fish.

According to some embodiments of the invention, the method further comprises (c) identifying the fish of the tilapia genus comprising the loss-of-function mutation in the slc45a2 gene.

According to some embodiments of the invention, the method further comprises (d) breeding the fish of step (b) or (c) with a second fish of the tilapia genus to produce a third fish of the tilapia genus having the loss-of-function mutation in the slc45a2 gene.

According to an aspect of some embodiments of the present invention there is provided a progeny of the fish of some embodiments of the invention.

According to an aspect of some embodiments of the present invention there is provided a population of fish comprising the fish of some embodiments of the invention, wherein the population is stable for the albino-red phenotype.

According to an aspect of some embodiments of the present invention there is provided a cell of the fish of some embodiments of the invention.

According to an aspect of some embodiments of the present invention there is provided a feed or food product comprising the fish of some embodiments of the invention.

According to some embodiments of the invention, the albino-red phenotype is devoid of black/grey pigmentation.

According to some embodiments of the invention, the albino-red phenotype comprises a red eye phenotype.

According to some embodiments of the invention, the loss-of-function mutation is heritable.

According to some embodiments of the invention, the fish and the second fish both carry at least one allele with a loss-of-function mutation in the slc45a2 gene.

According to some embodiments of the invention, the third fish is homozygous for the loss-of-function mutation in the slc45a2 gene.

According to some embodiments of the invention, the mutation is selected from the group consisting of a deletion, an insertion, a point mutation, an indel, and a combination thereof.

According to some embodiments of the invention, the mutation comprises two or more mutations in the slc45a2 gene.

According to some embodiments of the invention, the mutation is in a target sequence having a sequence selected from SEQ ID NO: 9, 10, 11 and 12 corresponding to the SEQ ID NO: 1.

According to some embodiments of the invention, the mutation is expressed in somatic cells.

According to some embodiments of the invention, the mutation is expressed in germline cells.

According to some embodiments of the invention, the fish of the tilapia genus is purebred.

According to some embodiments of the invention, the fish of the tilapia genus is a hybrid.

According to some embodiments of the invention, the fish of the tilapia genus is selected from the group consisting of Nile tilapia (Oreochromis niloticus), Blue tilapia (Oreochromis aureus), Mozambique tilapia (Oreochromis mossambicus), Wami tilapia (Oreochromis urolepis), Three spotted tilapia (Oreochromis andersonii), Longfin tilapia (Oreochromis macrochir), Sabaki tilapia (Oreochromis spilurus), Blackchin tilapia (Sarotherodon melanotheron), Mango tilapia (Sarotherodon galilaeus), Guinean tilapia (Coptodon guineensis), Redbreast tilapia (Coptodon rendalli) and Redbelly tilapia (Coptodon zillii).

According to some embodiments of the invention, introducing the DNA editing agent comprises introducing two or more DNA editing agents.

According to some embodiments of the invention, the two or more DNA editing agents target distinct sites within the slc45a2 gene.

According to some embodiments of the invention, the DNA editing agent comprises at least one gRNA.

According to some embodiments of the invention, the DNA editing agent comprises an endonuclease.

According to some embodiments of the invention, the endonuclease comprises Cas9.

According to some embodiments of the invention, the DNA editing agent comprises a DNA editing system selected from the group consisting of a CRISPR-endonuclease, a meganuclease, a zinc finger nucleases (ZFN), a transcription-activator like effector nuclease (TALEN), and a homing endonuclease.

According to some embodiments of the invention, the DNA editing agent is applied to the cell as DNA, RNA or RNP.

According to some embodiments of the invention, the DNA editing agent is linked to a reporter for monitoring expression in a fish cell.

According to some embodiments of the invention, the reporter is a fluorescent protein.

According to some embodiments of the invention, the loss-of-function mutation is determined genotypically.

According to some embodiments of the invention, the loss-of-function mutation is determined phenotypically.

According to some embodiments of the invention, the phenotype is determined prior to the genotype.

According to some embodiments of the invention, the genotype is determined prior to the phenotype.

According to some embodiments of the invention, the feed or food product being a whole fish, a fish portion, a fish meal or a fish oil.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B illustrate the evolutionary conservation of Nile tilapia slc45a2 gene. Phylogenetic tree (FIG. 1A) of various vertebrate slc45a2 mRNAs was constructed using Maximum Likelihood method based on the Jukes-Cantor model [Jukes, T. H. and Cantor, C. R. in Mammalian Protein Metabolism (ed. H. N. Munro) 21-132 (Academic Press, 1969)]. The percentage of trees in which the associated taxa clustered together is shown next to the branches. The tree is drawn to scale, with branch lengths measured in the number of substitutions per site. The analysis involved 23 nucleotide sequences. All positions containing gaps and missing data were eliminated. Evolutionary analyses were conducted in MEGA? [Kumar, S. et al., Mol Biol Evol (2016) 33, 1870-1874, doi:10.1093/molbev/msw054]. slc45a2 species and accession numbers used for this analysis were: Nile tilapia (Oreochromis niloticus; XM 003451484.3), Human (Homo sapiens; NM 016180.5), Dog (Canis lupus familiaris; NM 001037947.1), Zebrafish (Danio rerio; NM 001110377.1), Chicken (Gallus gallus; NM 001083364.2), Medaka (Oryzias latipes; NM 001104758.1), Mouse (Mus musculus; NM 053077.3), European eel (Anguilla anguilla; XM 035392013.1), Cat (Felis catus; NM 001127661.1), Sterlet (Acipenser ruthenus; XM 034014799.2), Northern pike (Esox lucius; XM 010877274.4), Giant grouper (Epinephelus lanceolatus; XM 033646335.1), Great tit (Parus major; XM 015615505.3), Sea lamprey (Petromyzon marinus; XM 032953415.1), Western clawed frog (Xenopus tropicalis; NM 001011335.1), Blue tilapia (Oreochromis aureus; XM 031754660.1), Gilt-head bream (Sparus aurata; XM 030435015.1), Sheep (Ovis aries; XM 004017064.3), Chinese alligator (Alligator sinensis; XM 006019120.3), Zebra mbuna (Maylandia zebra; XM 004549708.2), Marine madaka (Oryzias melastigma; XM 024299538.1), Yellowtail kingfish (Seriola lalandi dorsalis; XM 023408617.1), Ocellaris clownfish (Amphiprion ocellaris; XM 023264678.1); Conservation of chromosomal synteny was performed using Genomicus [Nguyen, Nga Thi T. et al., Nucleic Acids Research (2017) 46, D816-D822] (FIG. 1B). This analysis demonstrated moderate-high conservation of slc45a2 chromosomal neighboring genes. Gene names according to human nomenclature: SLC45A2—solute carrier family 45 member 2, RXFP3—relaxin family peptide receptor 3, PLPP1—phospholipid phosphatase 1, MTREX—Mtr4 exosome RNA helicase, DHX29—DExH-box helicase 29, ITGA1—integrin subunit alpha 1, PSTPIP2—proline-serine-threonine phosphatase interacting protein 2, GNG10—G protein subunit gamma 10, NXNL2—nucleoredoxin-like 2, BTC—betacellulin, epidermal growth factor family member, AMACR—alpha-methylacyl-CoA racemase, GSDF—gonadal somatic cell derived factor, KLHL8—kelch-like family member 8, SDAD1—SDA1 domain containing 1, MAPK10—mitogen-activated protein kinase 10, ARHGAP24—Rho GTPase activating protein 24, ESM1—endothelial cell specific molecule 1, ST6GALNAC6—ST6 (alpha-N-acetyl-neuraminyl-2,3-beta-galactosyl-1,3)-N-acetylgalactosaminide alpha-2,6-sialyltransferase 6, HCN1—potassium/sodium hyperpolarization-activated cyclic nucleotide-gated channel 1. Arrowhead direction indicates gene orientation relative to the slc45a2 strand.

FIGS. 2A-J illustrate transient analysis of slc45a2-RNPs activity in vivo. Nile tilapia zygotes were microinjected at single-cell stage with RNPs containing slc45a2-exon1 specific gRNAs. At 4 days post fertilization naïve embryos exhibited melanin formation in the eye and clear melanophores on the body and yolk surface (FIGS. 2A-C) while RNP injected embryos showed robust reduction to complete loss of melanin in the eye, body and yolk surface (FIGS. 2D-F). Sequence analysis demonstrated that three RNPs induced various genomic indels (FIG. 2G) including 21 nt insertion (grey highlighted) between two gRNA sites. gRNA target sequences are highlighted in bold italicized letters, PAM sequences are highlighted in red. Next-generation amplicon sequencing of the genomic target region confirmed the presence of variable levels of mutant alleles in all fish analyzed (FIG. 2H). Global mutagenic analysis demonstrated that most mutant alleles originated from the activity of gRNA3 alone or in combination with gRNA2, but not from gRNA2 activity alone (FIG. 2I). Variability analysis of gRNA-specific mutations demonstrated that gRNA3 activity resulted in significantly higher (p=0.021; Chi-square test) allele variation that was induced by gRNA2 activity (FIG. 2J).

FIGS. 3A-E illustrate phenotypic analysis of slc45a2-RNPs induced mutations in adult tilapia. Nile tilapia zygotes were microinjected at single-cell stage with RNPs containing slc45a2-exon1 specific gRNAs 2 and 3 (FIG. 3A). At 1 month post fertilization, naïve embryos exhibited normal grey-black pattern with dark eyes (FIG. 3B) while RNP injected fish showed approximately 97-99% loss of melanin in the skin and no melanin was seen in the eyes (FIG. 3C). This phenotype persisted post sexual maturation as F₀ mutant displayed almost complete lack of melanin (FIGS. 3D-E).

FIGS. 4A-C illustrate the molecular analysis of slc45a2-RNPs induced somatic and germline mutations in Nile tilapia. Genomic slc45a2-exon1 was amplified using gDNA extracted from F₀ fin-clip (FIG. 4A), F₀ sperm (FIG. 4B) and F₁ fin-clips (FIG. 4C) and cloned into pGEM®-T easy vector. Random colonies were sequenced and aligned according to the origin of template DNA. Of note, this analysis demonstrated that two of the six detected somatic alleles (FIG. 4A) and the seven detected sperm alleles (FIG. 4B) were identical. Of further note, 10 mutant germline alleles were detected in F₁ fish, including a 57 bp deletion between the two gRNA target sites (FIG. 4C). Identical allele colors indicate identical allelic sequence. Numbers on the right indicate the nature of indel, with “+” indicating nucleotide insertion and “—” indicating nucleotide deletion.

FIG. 5 illustrates aligned sequences of genomic slc45a2-exon1 from genomic database (chrLG7 (reverse strand): 16157420-16156969) and cloned region I of the analyzed Nile tilapia brood stock. SNPs are highlighted in yellow and gRNAs targeting to the SNP sites were avoided.

FIGS. 6A-D illustrate representative images of melanophore expression in WT (FIG. 6A) and heterozygote (FIG. 6B) scales. Scales of F2 homozygous mutant fish exhibited absence of pigmentation (FIG. 6C). Melanophore cell quantifications (melanophore cell count/scales area mm 2) showed no significant difference (unpaired t-test; p=0.252) in melanophore cell density between WT and heterozygous fish (FIG. 6D).

FIGS. 7A-F illustrate allele frequency and phenotypic analysis of slc45a2 mutants. HRM analysis of sequence validated slc45a2 mutant alleles in F₁ larvae showed that most alleles shared similar heredity level apart of the highly abundant allele 5 and the relatively rare allele of complete site-to-site deletion (FIG. 7A). Site-specific analysis demonstrated differential RNP activity with site 2 displaying higher indel variability (FIG. 7B) than site 3 (FIG. 7C), yet with a lower overall mutation rate. Phenotypic analysis of F₂ offspring demonstrated abundant melanin-expressing cells on the WT embryo surface at 3 days post fertilization (dpf) (FIG. 7D), while slc45a2^{-/-} mutants (FIG. 7D′) expressed no melanin. This phenotype became more pronounced at 5 dpf, when melanin accumulated also in the WT larval eye (FIG. 7E), whereas slc45a2 mutants exhibited complete OCA phenotype (FIG. 7F).

FIGS. 8A-B illustrate slc45a2 mutant phenotype in sexually mature fish. While WT fish (right-side fish in FIG. 8A) display grey coloration with characteristic black stripes and eye pigmentation, homozygous slc45a2 mutants show no melanin formation in their skin or eyes following sexual maturation as evident by their red eyes and solid albino-red to red skin coloration (left-side fish in FIG. 8A and the individually displayed fish in FIG. 8B).

### DESCRIPTION OF SPECIFIC EMBODIMENTS OF THE INVENTION

The present invention, in some embodiments thereof, relates to stable albino-red tilapia strains devoid of grey/black pigmentation and, more particularly, but not exclusively, to generation of same by gene editing.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

In recent years, there is an increasing demand for “red tilapia” which are commercial strains of hybrids of different tilapiine species (O. niloticus, O. mossambicus, O. aureus and O. hornorum, depending on the particular strain), or red variants of highly inbred Nile tilapia. Red tilapias have high demand and significantly increased market value relatively to the wild-type, which have grey to black colors. Yet, the red phenotype is genetically unstable and some of its regulating genes remain unknown. In wild-type tilapia, the red phenotype may be affected by environmental parameters, and results in non-uniform coloration with black or dark-red color blotches that reduce the market value of the fish. This emphasizes the need for a tilapia strain with a uniform red color that is stable throughout generations, and established on a clear genetic background that allows tractability of its inheritance under various breeding and genetic selection protocols.

While reducing the present invention to practice, the present inventors have generated a stable and heritable red tilapia phenotype by imparting a loss-of-function mutation in the slc45a2 gene.

As is shown herein below and in the Examples section which follows, the present inventors have identified and partially cloned the slc45a2 gene in Nile tilapia, and used these data to design highly specific guideRNAs (gRNA) for slc45a2 genomic sequence (Example 1, below). Multiplex microinjection of ribonucleoproteins containing recombinant-Cas9 protein and slc45a2 specific gRNAs to Nile tilapia zygotes induced various levels of melanin loss in the injected fish (Example 2, below). Next-generation amplicon sequencing (NGS) analysis demonstrated that all F₀ fish which developed from the injected zygotes carried mutated alleles in their genome at varied mutation levels (Example 2, below). One of the microinjected fish was a male that displayed approximately 97-99% loss of melanin in the skin (Example 3, below). Unlike the currently available red-tilapia lines, this line also displayed loss of eye melanin as reflected by its red eyes (Example 3, below). Sequencing of the genomic target region in the slc45a2 gene from gDNA extracted from fin-clip, sperm and offspring (F₁) of this male, identified various genomic indels and germline transduction of the sperm-identified indels (Example 3, below). Furthermore, high-resolution melt (HRM) analysis combined with sanger sequencing demonstrated the surprising and unexpected presence of 10 heritable alleles in F₁ (Example 4, below). Moreover, second generation (F₂) slc45a2^{-/-} tilapia displayed a clear oculocutaneous albinism (OCA) phenotype, i.e. complete loss of melanin in the eyes and skin resulting in a red phenotype (Example 4, below).

Taken together, albino-red tilapia fish were generated comprising somatic and germline slc45a2 mutant alleles which are both stable and genetically trackable. These fish displayed red phenotype of the skin and eyes which was stable in the fish population and hereditary.

Thus, according to one aspect of the present invention there is provided a fish of a tilapia genus comprising a loss-of-function mutation in a slc45a2 gene, wherein the mutation is in a homozygous form, and wherein the loss-of-function mutation results in an albino-red phenotype of the fish.

The term “fish of a tilapia genus” as used herein refers to a member of the group of the tilapiine cichlids. Exemplary members of the tilapia genus include, but are not limited to, Nile tilapia (Oreochromis niloticus), Blue tilapia (Oreochromis aureus), Mozambique tilapia (Oreochromis mossambicus), Wami tilapia (e.g. the two subspecies: Oreochromis urolepis urolepis and Oreochromis urolepis hornorum), Three spotted tilapia (Oreochromis andersonii), Longfin tilapia (Oreochromis macrochir), Sabaki tilapia (Oreochromis spilurus), Blackchin tilapia (Sarotherodon melanotheron), Mango tilapia (Sarotherodon galilaeus), Guinean tilapia (Coptodon guineensis), Redbreast tilapia (Coptodon rendalli) and Redbelly tilapia (Coptodon zillii), or hybrids thereof.

The fish of the tilapia genus of the invention can be of any age (e.g. fish fry, juveniles, fingerlings, or adult/mature fish). Furthermore, any fish aquaculture techniques known in the art can be used to stock, maintain, reproduce, and gather the fish used in the invention, as further discussed below.

The “wild-type” tilapia fish comprise a wild-type sequence of the slc45a2 gene.

Wild-type (WT) strains of tilapia genus may present black/grey to red/pink/blonde coloration.

According to some embodiments, the wild-type strains of tilapia presenting a black/grey phenotype typically display black and/or grey pigmentation on the skin and the peritoneum, and black eyes. Black to grey coloration in tilapia is usually associated with solid color appearance.

According to some embodiments, the wild-type strains of tilapia presenting a red phenotype (referred to as Red tilapia) lack most of the black and/or grey skin pigmentation found in black/grey tilapia, yet typically exhibit black/grey blotching (e.g. spots, e.g. blotches) on their skin, black/grey peritoneum, and black eye coloration.

According to some embodiments, the wild-type strains of tilapia presenting a blonde-pink phenotype (referred to as Blonde/Pink tilapia) typically lack pigmentation on the skin yet may exhibit dark blotches on their skin (e.g. pink with scattered red spots or pink with scattered black spots) and typically exhibit black eye coloration.

As mentioned, the fish of the tilapia genus of the invention comprise an albino-red phenotype and are genetically modified.

The term “albino-red phenotype” as used herein refers to tilapia displaying a loss of melanin in the skin, peritoneum and eyes. Accordingly, the albino-red tilapia of the invention lack black/grey pigmentation or blotching on the skin and peritoneum (i.e. are completely devoid of black/grey pigmentation or black/grey blotches). The albino-red fish may exhibit red blotches on their skin or peritoneum. Furthermore, the albino-red tilapia fish of the invention display red eye phenotype (due to complete loss of melanin).

The albino-red phenotype shows no black/grey pigmentation on their skin and peritoneum due to lack of pigmented melanophores on their body, and shows no melanin in their eyes. Determining loss of black/grey pigments can be carried out using any method known in the art, e.g. by binoculars, under a microscope, by image analysis or by digital photography. Additional methodologies for assessing fish coloration are further discussed in Svensson and Skold, “Skin Biopsies as Tools to Measure Fish Coloration and Colour Change” (2011) In book: Skin Biopsy—Perspectives, incorporated herein by reference.

As used herein the term “slc45a2 gene” refers to the gene encoding the Solute Carrier Family 45 Member 2 protein having the GeneBank Accession nos. XP_003451532 (protein) and XM_003451484 (mRNA), or homologs thereof. Slc45a2 is also referred to as membrane-associated transporter protein (MATP), absent in melanoma-1 (Aim-1), oculocutaneous albinism type 4 (OCA4), and B gene and as albino (alb).

According to one embodiment, slc45a2 gene homolog refers to a gene encoding the slc45a2 protein in different tilapia strains.

The fish of the invention comprise a loss-of-function mutation in a slc45a2 gene.

As used herein, the phrase “loss-of-function mutation” refers to any mutation in the DNA sequence of a gene (e.g., slc45a2 gene) which results in downregulation of the expression level and/or activity of the expressed product, i.e., the mRNA transcript and/or the translated protein. Non-limiting examples of such loss-of-function mutations include a missense mutation, i.e., a mutation which changes an amino acid residue in the protein with another amino acid residue and thereby abolishes the activity of the protein; a nonsense mutation, i.e., a mutation which introduces a stop codon in a protein, e.g., an early stop codon which results in a shorter protein devoid of the activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the activity), or in a longer amino acid sequence (e.g., a read-through protein) which affects the secondary or tertiary structure of the protein and results in a non-functional protein, devoid of the activity of the non-mutated polypeptide; a read-through mutation due to a frame-shift mutation or a modified stop codon mutation (i.e., when the stop codon is mutated into an amino acid codon), with an abolished activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to a specific embodiment, loss-of-function mutation in slc45a2 gene results in a gene product which does not encode a functional slc45a2 protein, i.e. functional means that it mediates melanin synthesis in tilapia.

According to specific embodiments loss-of-function mutation of a gene may comprise at least one allele of the gene.

According to other specific embodiments loss-of-function mutation of a gene comprises both alleles of the gene. In such instances the loss-of-function mutation in the slc45a2 gene may be in a homozygous form. According to this embodiment, homozygosity is a condition where both alleles at the slc45a2 gene locus are characterized by a loss-of-function mutation. Heterozygosity refers to a condition wherein one of the alleles at the slc45a2 gene locus is characterized by a loss-of-function mutation.

According to one embodiment, the loss-of-function mutation is in a homozygous form. It will be appreciated that the mutation in each of the two alleles may be identical or different (e.g. insertion, deletion, indel, as discussed below). In addition, the mutation in each of the two alleles may be in the same or in different positions on the slc45a2 gene locus. Regardless of the mutation or position thereof in each of the two alleles, the mutation in each of the two alleles results a loss-of-function mutation.

According to one embodiment, the loss-of-function mutation is heritable, i.e. is transmissible from parent to offspring.

Methods of introducing nucleic acid alterations to a gene of interest are well known in the art [see for example Menke D. Genesis (2013) 51: -618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed by publically available sources or obtained commercially from Transposagen, Addgene and Sangamo Biosciences.

According to one aspect of the invention, there is provided a method of generating the fish of some embodiments of the invention, the method comprising: (a) introducing into a zygote of the fish of the tilapia genus a DNA editing agent conferring a loss-of-function mutation in the slc45a2 gene; and (b) allowing the zygote of step (a) to develop into a fish.

Following is a description of various non-limiting examples of methods and DNA editing agents used to introduce nucleic acid alterations to a nucleic acid sequence (genomic) of slc45a2 and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR.

Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs), T-GEE system and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break (DSB). Repair of these double-stranded breaks through the nonhomologous end-joining (NHEJ) pathway most often results in small deletions or small sequence insertions (Indels). Since each repair made by NHEJ is unique, the use of a single nuclease pair can produce an allelic series with a range of different deletions at the target site.

The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2—His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence (gRNA), and a trans-activating crRNA (tracrRNA) (Jinek et al. Science (2012) 337: 816-821.).

It was further demonstrated that a synthetic chimeric single guide RNA (sgRNA) composed of a fusion between crRNA and tracrRNA could direct Cas9 to cleave DNA targets that are complementary to the crRNA in vitro. It was also demonstrated that transient expression of Cas9 in conjunction with synthetic sgRNAs can be used to produce targeted double-stranded breaks (DSBs) in a variety of different species (Cho et al., 2013; Cong et al., 2013; DiCarlo et al., 2013; Hwang et al., 2013a,b; Jinek et al., 2013; Mali et al., 2013). The sgRNA (also referred to herein as single guide RNA (sgRNA)) is typically 80-100-nucleotide sequence encoding a combination of the target homologous sequence (crRNA) and the endogenous bacterial RNA that links the crRNA to the Cas9 nuclease (tracrRNA) in a single chimeric transcript.

The CRIPSR/Cas system for genome editing contains two distinct components: a sgRNA and an endonuclease e.g. Cas9, or three distinct components a gRNA, a tracrRNA and an endonuclease e.g. Cas9.

The sgRNA/Cas9 complex or the gRNA/tracrRNA/Cas9 is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the sgRNA/Cas9 complex or of the gRNA/tracrRNA/Cas9 localizes the Cas9 to the genomic target sequence so that the Cas9 can cut both strands of the DNA causing a double-strand break (DSB). Just as with ZFNs and TALENs, the double-stranded breaks (DSBs) produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system is coupled with the ability to easily create synthetic sgRNAs or gRNAs. This creates a system that can be readily modified to target modifications at different genomic sites and/or to target different modifications at the same site (e.g. in the slc45a2 gene locus). Additionally, protocols have been established which enable simultaneous targeting of multiple genes. The majority of cells carrying the mutation present biallelic mutations in the targeted genes.

However, apparent flexibility in the base-pairing interactions between the sgRNA or the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is mostly repaired by single strand break repair mechanism involving proteins such as but not only, PARP (sensor) and XRCC1/LIG III complex (ligation). However, two proximal, opposite strand nicks introduced by a Cas9 nickase are treated as a double-strand break, in what is often referred to as a ‘double nick’ CRISPR system. A double-nick, which is basically non-parallel DSB, can be repaired like other DSBs by HR or NHEJ depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that are not likely to change the genomic DNA.

Modified versions of the Cas9 enzyme containing two inactive catalytic domains (dead Cas9, or dCas9) have no nuclease activity while still able to bind to DNA based on sgRNA or gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

Alternatively, CRISPR systems may be fused with various effector domains, such as DNA cleavage domains. The DNA cleavage domain can be obtained from any endonuclease or exonuclease. Non-limiting examples of endonucleases from which a DNA cleavage domain can be derived include, but are not limited to, restriction endonucleases and homing endonucleases (see, for example, New England Biolabs Catalog or Belfort et al. (1997) Nucleic Acids Res.), e.g. Fokl endonuclease and I-CreI.

Additional Cas endonucleases that can be used to effect DNA editing with gRNA include, but are not limited to, Cas9, Cpf1 (Zetsche et al., 2015, Cell. 163(3):759-71), C2c1, C2c2, C2c3 (Shmakov et al., Mol Cell. 2015 Nov. 5; 60(3):385-97), CasX and Cpf1/Cas12a.

There are a number of publicly available tools available to help choose and/or design target sequences as well as lists of bioinformatically determined unique sgRNAs or gRNA for different genes in different species such as, but not limited to, the Feng Zhang lab's Target Finder, The Alex Scier Lab's Target Finder (ChopChop), the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, crRNA (gRNA), tracrRNA and a Cas endonuclease (e.g. Cas9) should be expressed or present (e.g., as a ribonucleoprotein complex (RNP)) in a target cell. Alternatively, both sgRNA and a Cas endonuclease (e.g. Cas9), or the gRNA, tracrRNA and a Cas endonuclease (e.g. Cas9), should be expressed or present (e.g., as a ribonucleoprotein complex) in a target cell. The insertion vector can contain all cassettes on a single plasmid or the cassettes are expressed from separate plasmids. CRISPR plasmids are commercially available such as the px330 plasmid from Addgene (Cambridge, Mass.).

According to a specific embodiment, the DNA editing agent comprises a DNA targeting module (e.g., gRNA).

According to a specific embodiment, the DNA editing agent comprises two or more DNA targeting modules (e.g., 2, 3, 4, 5 or more different gRNAs targeting different regions within the slc45a2 gene locus).

According to a specific embodiment, the DNA editing agent comprises a nuclease (e.g. an endonuclease) and a DNA targeting module (e.g., gRNA, or gRNA and tracrRNA).

According to a specific embodiment, the DNA editing agent comprises a nuclease (e.g. an endonuclease) and a DNA targeting module (e.g., sgRNA).

According to a specific embodiment, the DNA editing agent is CRISPR/endonuclease.

According to a specific embodiment, the DNA editing agent is CRISPR/Cas, e.g. sgRNA and Cas9 or a gRNA, tracrRNA and dCas9.

Non-limiting examples of gRNAs that can be used in the present invention comprise a nucleic acid sequence as set forth in SEQ ID NOs: 13, 14 and 15 (i.e. gRNA1, gRNA2 and gRNA3, respectively).

According to one embodiment, gRNAs that can be used in the present invention comprise a combination of the gRNAs having the nucleic acid sequence as set forth in SEQ ID NOs: 13, 14 and 15 (e.g. gRNA2 and gRNA3, gRNA1 and gRNA3, gRNA1 and gRNA2 or gRNA1, gRNA2 and gRNA3).

According to a specific embodiment, the CRISPR comprises a short guide RNA (sgRNA) comprising a nucleic acid sequence as set forth in SEQ ID NO: 16.

According to a specific embodiment, the gRNA targets slc45a2-exon1 genomic region (accession no. XM_003451484), e.g. as set forth in SEQ ID NO: 46-48 (i.e. genomic slc45a2 from genomic database (chrLG7 (reverse strand): 16157420-16156969).

Additional DNA editing agents and systems which may be used to introduce nucleic acid alterations to a nucleic acid sequence (genomic) of slc45a2 according to the present teachings include, but are not limited to, transposons and TFOs. These are discussed briefly below.

Transposon—refers to a mobile genetic element comprising a nucleotide sequence which can move around to different positions within the genome of a single cell. In the process the transposon can cause mutations and/or change the amount of a DNA in the genome of the cell. A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner.

Triplex forming oligonuclotides (TFOs)—TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was shown that synthetic oligonucleotides can be targeted to specific sequences (see Seidman and Glazer, J Clin Invest (2003) 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression.

Additionally, TFOs designed according to the abovementioned principles can induce directed mutagenesis capable of effecting DNA repair, thus providing both downregulation and upregulation of expression of endogenous genes (Seidman and Glazer, J Clin Invest (2003) 112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

It will be appreciated that the DNA editing agent can be a mutagen that causes random mutations and the cells exhibiting downregulation of the expression level of slc45a2 may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG), ethyl methane sulfonate (EMS) or N-ethyl-N-nitrosourea (ENU).

Regardless of the DNA editing agent or method used, the method of the invention is employed such that the slc45a2 gene is modified by at least one of a deletion, an insertion, a point mutation or an indel.

Regardless of the method employed to introduce nucleic acid alterations to a nucleic acid sequence (genomic) of slc45a2, the method is employed such that a mutation is introduced in at least one position on the slc45a2 gene locus (e.g. in 1, 2, 3, 4, 5 or more distinct positions on the slc45a2 gene locus).

According to one embodiment, the modification imparts a loss-of-function mutation (as discussed above).

According to one embodiment, mutation is in a target sequence having a sequence selected from SEQ ID NO: 9, 10, 11 and 12 corresponding to SEQ ID NO: 1.

According to one embodiment, the mutation is in one position in the slc45a2 gene locus.

According to one embodiment, the mutation is in two or more positions in the slc45a2 gene locus (e.g. in 2, 3, 4, 5, or more positions in the slc45a2 gene locus).

According to a specific embodiment, the modification (e.g. insertion, deletion, point mutation and/or indel) comprises a modification of about 1-100,000 nucleotides, about 1-50,000 nucleotides, about 1-25,000 nucleotides, about 1-10,000 nucleotides, about 1-7,500 nucleotides, about 1-5,000 nucleotides, about 1-2,500 nucleotides, about 1-2,000 nucleotides, about 1-1,000 nucleotides, about 1-750 nucleotides, about 1-500 nucleotides, about 1-250 nucleotides, about 1-200 nucleotides, about 1-150 nucleotides, about 1-100 nucleotides, about 1-90 nucleotides, about 1-80 nucleotides, about 1-70 nucleotides, about 1-60 nucleotides, about 1-50 nucleotides, about 1-nucleotides, about 1-30 nucleotides, about 1-25 nucleotides, about 1-20 nucleotides, about 1-nucleotides, about 1-10 nucleotides, about 1-5 nucleotides (as compared to the wild type slc45a2 gene).

According to one embodiment, the modification (e.g. insertion, deletion, point mutation and/or indel) comprises a modification of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 70, 80, 90, 100, 110, 120, 130, 140, 150, 160, 170, 180, 190, 200, 250, 300, 350, 400, 450, 500, 750, 1000, 1500, 2000, 2500, 5000, 7500 or nucleotides (as compared to the wild type slc45a2 gene).

According to a specific embodiment, the modification (e.g. insertion, deletion, point mutation and/or indel) comprises a modification of 1 nucleotide.

According to a specific embodiment, the modification (e.g. insertion, deletion, point mutation and/or indel) comprises a modification of about 5 nucleotides.

According to a specific embodiment, the modification (e.g. insertion, deletion, point mutation and/or indel) comprises a modification of about 10 nucleotides.

According to a specific embodiment, the modification (e.g. insertion, deletion, point mutation and/or indel) comprises a modification of about 20 nucleotides.

According to a specific embodiment, the modification (e.g. insertion, deletion, point mutation and/or indel) comprises a modification of about 30 nucleotides.

According to a specific embodiment, the modification (e.g. insertion, deletion, point mutation and/or indel) comprises a modification of about 50 nucleotides.

According to a specific embodiment, the modification (e.g. insertion, deletion, point mutation and/or indel) comprises a modification of about 60 nucleotides.

According to a specific embodiment, the modification (e.g. insertion, deletion, point mutation and/or indel) comprises a modification of up to 5 nucleotides.

According to a specific embodiment, the modification (e.g. insertion, deletion, point mutation and/or indel) comprises a modification of up to 10 nucleotides.

According to a specific embodiment, the modification (e.g. insertion, deletion, point mutation and/or indel) comprises a modification of up to 15 nucleotides.

According to a specific embodiment, the modification (e.g. insertion, deletion, point mutation and/or indel) comprises a modification of up to 20 nucleotides.

According to a specific embodiment, the modification (e.g. insertion, deletion, point mutation and/or indel) comprises a modification of up to 25 nucleotides.

According to a specific embodiment, the modification (e.g. insertion, deletion, point mutation and/or indel) comprises a modification of up to 50 nucleotides.

According to a specific embodiment, the modification (e.g. insertion, deletion, point mutation and/or indel) comprises a modification of up to 60 nucleotides.

According to a specific embodiment, the modification (e.g. insertion, deletion, point mutation and/or indel) comprises a modification of up to 70 nucleotides.

According to a specific embodiment, the modification (e.g. insertion, deletion, point mutation and/or indel) comprises a modification of up to 80 nucleotides.

According to a specific embodiment, the modification (e.g. insertion, deletion, point mutation and/or indel) comprises a modification of up to 90 nucleotides.

According to a specific embodiment, the modification (e.g. insertion, deletion, point mutation and/or indel) comprises a modification of up to 100 nucleotides.

According to a specific embodiment, the modification (e.g. insertion, deletion, point mutation and/or indel) comprises a modification of up to 250 nucleotides.

According to a specific embodiment, the modification (e.g. insertion, deletion, point mutation and/or indel) comprises a modification of up to 500 nucleotides.

According to a specific embodiment, the modification (e.g. insertion, deletion, point mutation and/or indel) comprises a modification of up to 1000 nucleotides.

According to one embodiment, the modification (e.g. insertion, deletion, point mutation and/or indel) comprises a modification of at most 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 70, 80, 90, 100, 110, 120, 130, 140, 150, 160, 170, 180, 190, 200, 250, 300, 350, 400, 450, 500, 750, 1000, 1500, 2000, 2500, 5000, 7500, or at most 10,000 nucleotides (as compared to the wild type slc45a2 gene).

According to a specific embodiment, the modification (e.g. insertion, deletion, point mutation and/or indel) comprises a modification of at most 1000 nucleotides.

According to a specific embodiment, the modification (e.g. insertion, deletion, point mutation and/or indel) comprises a modification of at most 750 nucleotides.

According to a specific embodiment, the modification (e.g. insertion, deletion, point mutation and/or indel) comprises a modification of at most 500 nucleotides.

According to a specific embodiment, the modification (e.g. insertion, deletion, point mutation and/or indel) comprises a modification of at most 250 nucleotides.

According to a specific embodiment, the modification (e.g. insertion, deletion, point mutation and/or indel) comprises a modification of at most 100 nucleotides.

According to a specific embodiment, the modification (e.g. insertion, deletion, point mutation and/or indel) comprises a modification of at most 90 nucleotides.

According to a specific embodiment, the modification (e.g. insertion, deletion, point mutation and/or indel) comprises a modification of at most 80 nucleotides.

According to a specific embodiment, the modification (e.g. insertion, deletion, point mutation and/or indel) comprises a modification of at most 70 nucleotides.

According to a specific embodiment, the modification (e.g. insertion, deletion, point mutation and/or indel) comprises a modification of at most 60 nucleotides.

According to a specific embodiment, the modification (e.g. insertion, deletion, point mutation and/or indel) comprises a modification of at most 50 nucleotides.

According to a specific embodiment, the modification (e.g. insertion, deletion, point mutation and/or indel) comprises a modification of at most 40 nucleotides.

According to a specific embodiment, the modification (e.g. insertion, deletion, point mutation and/or indel) comprises a modification of at most 30 nucleotides.

According to a specific embodiment, the modification (e.g. insertion, deletion, point mutation and/or indel) comprises a modification of at most 25 nucleotides.

According to a specific embodiment, the modification (e.g. insertion, deletion, point mutation and/or indel) comprises a modification of at most 20 nucleotides.

According to a specific embodiment, the modification (e.g. insertion, deletion, point mutation and/or indel) comprises a modification of at most 15 nucleotides.

According to a specific embodiment, the modification (e.g. insertion, deletion, point mutation and/or indel) comprises a modification of at most 10 nucleotides.

According to a specific embodiment, the modification (e.g. insertion, deletion, point mutation and/or indel) comprises a modification of at most 5 nucleotides.

According to a specific embodiment, the modification (e.g. insertion, deletion, point mutation and/or indel) comprises a modification in one allele of the slc45a2 gene.

According to a specific embodiment, the modification (e.g. insertion, deletion, point mutation and/or indel) comprises a modification in both alleles of the slc45a2 gene. Accordingly, the modification may comprise an insertion in both alleles, a deletion in both alleles, a point mutation in both alleles or an indel in both alleles. Alternatively, the mutation may comprise an insertion in one allele and a deletion, point mutation or indel in the other allele, a deletion in one allele and an insertion, point mutation or indel in the other allele, a point mutation in one allele and an insertion, deletion or indel in the other allele, or an indel in one allele and an insertion, deletion, or point mutation in the other allele.

According to a specific embodiment, the modification comprises a deletion of 57 nucleotides of the slc45a2 gene (as set forth in SEQ ID NO: 1).

According to a specific embodiment, the modification comprises a deletion of the entire gene (i.e. slc45a2 gene).

According to a specific embodiment, the modification comprises a chromosomal deletion of the entire slc45a2 gene.

Methods for qualifying efficacy and detecting sequence alteration are well known in the art and include, but not limited to, DNA sequencing, next-generation amplicon sequencing (NGS), electrophoresis, an enzyme-based mismatch detection assay and a hybridization assay such as PCR, RT-PCR, High-Resolution Melt curve (HRM), RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

According to one embodiment, the DNA editing agents (e.g. gRNA or sgRNA, or vector encoding thereof) can include (e.g. be linked to) at least one reporter that allows transformed cells containing the reporter to be either recovered by negative selection (by inhibiting the growth of cells that do not contain the reporter), or by positive selection (by screening for the product encoded by the reporter).

According to one embodiment, the reporter is a fluorescent reporter protein.

The term “a fluorescent protein” refers to a polypeptide that emits fluorescence and is typically detectable by flow cytometry, microscopy or any fluorescent imaging system, therefore can be used as a basis for selection of cells expressing such a protein.

Examples of fluorescent proteins that can be used as markers are, without being limited to, the Green Fluorescent Protein (GFP), the Blue Fluorescent Protein (BFP) and the red fluorescent proteins (e.g. dsRed, mCherry, RFP).

For example, GFP from the jellyfish Aequorea victoria, produces fluorescence upon exposure to ultraviolet light without the addition of a substrate (Chalfie et al., Science 263:802-5 (1994)). A number of modified GFPs have been created that generate as much as 50-fold greater fluorescence than does wild type GFP under standard conditions (Cormack et al., Gene 173:33-8 (1996); Zolotukhin et al., J. Virol 70:4646-54 (1996)). This level of fluorescence allows the detection of low levels of tissue specific expression in a living animal.

A review of new classes of fluorescent proteins and applications can be found in Trends in Biochemical Sciences [Rodriguez, Erik A.; Campbell, Robert E.; Lin, John Y; Lin, Michael Z.; Miyawaki, Atsushi; Palmer, Amy E.; Shu, Xiaokun; Zhang, Jin; Tsien, Roger Y. “The Growing and Glowing Toolbox of Fluorescent and Photoactive Proteins”. Trends in Biochemical Sciences. Doi: 10.1016/j.tibs.2016.09.010].

According to one embodiment, the reporter is an enzyme, such as (3-galactosidase, luciferase, and alkaline phosphatase, that can produce specific detectable products, and proteins that can be directly detected

According to one embodiment, the reporter is a synthetic dye, e.g. Cyanine. Cyanine dyes have the general formula: R2N[CH═CH]nCH═N+R2↔R2N+=CH[CH═CH]nNR2 (n is a small number) and are typically synthesized from 2, 3, 5 or 7-methine structures with reactive groups on either one or both of the nitrogen ends so that they can be chemically linked to either nucleic acids or protein molecules. Exemplary Cyanine dyes include, without being limited to, Cy2, Cy3, Cy5, and Cy7.

The disclosed fish of the tilapia genus of some embodiments of the invention are generated by introducing the DNA editing agent into cells of a fish, preferably embryonic cells, and most preferably in a single cell embryo (e.g. zygote, i.e. egg cell after fertilization with a sperm). Where the DNA editing agent is introduced into embryonic cells, the fish of the tilapia genus is obtained by allowing the embryonic cell or cells to develop into a fish (as discussed in detail below). Introduction of the DNA editing agent into embryonic cells of fish, and subsequent development of the fish, are simplified by the fact that embryos develop outside of the parent fish in most fish species.

According to one embodiment, the method is effected by introducing into cells of a fish, e.g. embryonic cells (e.g. zygote), a DNA editing agent.

According to one embodiment, the method is effected by introducing into cells of a fish, e.g. embryonic cells (e.g. zygote), two or more DNA editing agents (e.g., 2, 3, 4, 5 or more different DNA editing agents).

According to one embodiment, the two or more DNA editing agents target distinct sites within the slc45a2 gene locus.

According to a specific embodiment, the method is effected by introducing into cells of a fish, e.g. embryonic cells (e.g. zygote), a gRNA having the nucleic acid sequence as set forth in SEQ ID NOs: 13, 14 and 15 (i.e. gRNA1, gRNA2 and gRNA3, respectively).

According to a specific embodiment, the method is effected by introducing into cells of a fish, e.g. embryonic cells (e.g. zygote), two or more gRNAs having the nucleic acid sequence as set forth in SEQ ID NOs: 13, 14 and 15 (e.g. co-introducing gRNA2 and gRNA3, gRNA1 and gRNA3, gRNA1 and gRNA2, or gRNA1, gRNA2 and gRNA3).

According to a specific embodiment, the method is effected by introducing into cells of a fish, e.g. embryonic cells (e.g. zygote), a sgRNA having the nucleic acid sequence as set forth in SEQ ID NO: 16.

The DNA editing agent of the invention may be introduced into cells of a fish of the tilapia genus (e.g. into a zygote of the fish) using DNA delivery methods (e.g. by expression vectors) or using DNA-free methods.

According to one embodiment, the gRNA or sgRNA (or any other DNA recognition module used, dependent on the DNA editing system that is used) can be provided as RNA to the cell.

Thus, it will be appreciated that the present techniques relate to introducing the DNA editing agent using transient DNA or DNA-free methods such as RNA transfection (e.g. mRNA+sgRNA transfection), or Ribonucleoprotein (RNP) transfection (e.g. protein-RNA complex transfection, e.g. Cas9/sgRNA ribonucleoprotein (RNP) complex transfection).

For example, Cas9 can be introduced as a DNA expression plasmid, in vitro transcript (i.e. RNA), or as a recombinant protein bound to the RNA portion in a ribonucleoprotein particle (RNP). sgRNA, for example, can be delivered either as a DNA plasmid or as an in vitro transcript (i.e. RNA).

Any method known in the art for RNA or RNP transfection can be used in accordance with the present teachings, such as, but not limited to microinjection [as described by Cho et al., “Heritable gene knockout in Caenorhabditis elegans by direct injection of Cas9-sgRNA ribonucleoproteins,” Genetics (2013) 195:1177-1180, incorporated herein by reference], electroporation [as described by Kim et al., “Highly efficient RNA-guided genome editing in human cells via delivery of purified Cas9 ribonucleoproteins” Genome Res. (2014) 24:1012-1019, incorporated herein by reference], or lipid-mediated transfection e.g. using liposomes [as described by Zuris et al., “Cationic lipid-mediated delivery of proteins enables efficient protein-based genome editing in vitro and in vivo” Nat Biotechnol. (2014) doi: 10.1038/nbt.3081, incorporated herein by reference]. Additional methods of RNA transfection are described in U.S. Patent Application No. 20160289675, incorporated herein by reference in its entirety.

One advantage of RNA transfection methods of the invention is that RNA transfection is essentially transient and vector-free. An RNA transgene can be delivered to a cell and expressed therein, as a minimal expressing cassette without the need for any additional sequences (e.g. viral sequences).

According to one embodiment, for expression of exogenous DNA editing agents of the invention in cells, a polynucleotide sequence encoding the DNA editing agent is ligated into a nucleic acid construct suitable for cell expression. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration in eukaryotes (e.g., shuttle vectors). In addition, typical cloning vectors may also contain a transcription and translation initiation sequence, an expression sequence, a transcription and translation terminator and a polyadenylation signal.

Expression sequences are divided into two main classes, promoters and enhancers. A promoter is generally a sequence or sequences of DNA that function when in a relatively fixed location in regard to the transcription start site. A promoter contains core elements required for basic interaction of RNA polymerase and transcription factors, and may contain upstream elements and response elements. Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized in generation of the disclosed fish is active in the specific cell population transformed. For expression in a fish cell, the promoter employed can be a constitutive promoter, a tissue specific promoter, an inducible promoter, a chimeric promoter or a developmentally regulated promoter. Examples of preferred promoters useful for the methods of some embodiments of the invention include, but are not limited to, VASA, EF1α, β-actin, U6, CMV.

Enhancer generally refers to a sequence of DNA that functions at no fixed distance from the transcription start site and can be in either orientation. Enhancers function to increase transcription from nearby promoters. Enhancers also often contain response elements that mediate the regulation of transcription. Promoters can also contain response elements that mediate the regulation of transcription.

Enhancers often determine the regulation of expression of a gene. This effect has been seen in so-called enhancer trap constructs where introduction of a construct containing a reporter gene operably linked to a promoter is expressed only when the construct inserts into the domain of an enhancer (O'Kane and Gehring, Proc. Natl. Acad. Sci. USA 84:9123-9127 (1987), Allen et al., Nature 333:852-855 (1988), Kothary et al., Nature 335:435-437 (1988), Gossler et al., Science 244:463-465 (1989)). In such cases, the expression of the construct is regulated according to the pattern of the newly associated enhancer. Constructs having only a minimal promoter can be used in the disclosed fish to identify enhancers.

As mentioned, reporter proteins are useful for detecting or quantitating expression from expression sequences. For example, operatively linking nucleotide sequence encoding a reporter protein to a tissue specific expression sequences allows one to carefully study lineage development. Many reporter proteins are known and have been used for similar purposes in other organisms. These include fluorescent proteins and enzymes, as discussed above.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

It will be appreciated that the individual elements comprised in the expression vector can be arranged in a variety of configurations. For example, enhancer elements, promoters and the like, and even the polynucleotide sequence(s) encoding a DNA editing agent can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for fish expression vectors include, but are not limited to, pCDNA3.1, pCDNA6, Tol2kit plasmids, pSC2 which are available e.g. from Addgene, Invitrogen, Kawakami Lab and Chien lab.

According to one embodiment, in order to express a functional DNA editing agent, in cases where the cleaving module (nuclease) is not an integral part of the DNA recognition unit, the expression vector may encode the cleaving module as well as the DNA recognition unit (e.g. gRNA or sgRNA in the case of CRISPR/Cas).

Alternatively, the cleaving module (nuclease) and the DNA recognition unit (e.g. gRNA or sgRNA) may be cloned into separate expression vectors. In such a case, at least two different expression vectors must be transformed into the same eukaryotic cell.

Alternatively, when a nuclease is not utilized (i.e. not administered from an exogenous source to the cell), the DNA recognition unit (e.g. gRNA or sgRNA) may be cloned and expressed using a single expression vector.

According to one embodiment, the DNA editing agent comprises a nucleic acid agent encoding at least one DNA recognition unit (e.g. gRNA or sgRNA) operatively linked to a cis-acting regulatory element active in eukaryotic cells (e.g., promoter).

According to one embodiment, the nuclease (e.g. Cas9) and the DNA recognition unit (e.g. gRNA or sgRNA) are encoded from the same expression vector. Such a vector may comprise a single cis-acting regulatory element active in eukaryotic cells (e.g., promoter) for expression of both the nuclease and the DNA recognition unit. Alternatively, the nuclease and the DNA recognition unit may each be operably linked to a cis-acting regulatory element active in eukaryotic cells (e.g., promoter).

According to one embodiment, the nuclease (e.g. e.g. Cas9) and the DNA recognition unit (e.g. gRNA or sgRNA) are encoded from different expression vectors whereby each is operably linked to a cis-acting regulatory element active in eukaryotic cells (e.g., promoter).

The disclosed constructs can be introduced into embryonic fish cells (e.g. into zygotes) using any suitable technique. Many techniques for such introduction of exogenous genetic material have been demonstrated in fish and other animals. These include microinjection (described by, for example, Culp et al. (1991)), electroporation (described by, for example, Inoue et al., Cell. Differ. Develop. 29:123-128 (1990); Muller et al., FEBS Lett. 324:27-32 (1993); Murakami et al., J. Biotechnol. 34:35-42 (1994); Muller et al., Mol. Mar. Biol. Biotechnol. 1:276-281 (1992); and Symonds et al., Aquaculture 119:313-327 (1994)), particle gun bombardment (Zelenin et al., FEBS Lett. 287:118-120 (1991)), and the use of liposomes (Szelei et al., Transgenic Res. 3:116-119 (1994)). Microinjection is preferred. The preferred method for introduction of constructs into fish embryonic cells by microinjection is described in the examples.

Embryos or embryonic cells of fish of the tilapia genus can generally be obtained by collecting eggs immediately after they are laid. Depending on the type of fish, it is generally preferred that the eggs be fertilized prior to or at the time of collection. This is preferably accomplished by placing a male and 4-5 female fish together in a tank that allows egg collection. A fertilized egg cell prior to the first cell division is considered a one cell embryo, and the fertilized egg cell is thus considered an embryonic cell.

After introduction of the DNA editing agent, the embryo is allowed to develop into a fish. This is typically carried out by incubating the embryos under the same conditions used for incubation of eggs, e.g. at 27° C. under constant agitation. If appropriate, expression of an introduced construct can be observed during development of the embryo. At any step along the way the fish can be elected genotypically or phenotypically or both.

Fish harboring a loss-of-function mutation in slc45a2 gene can be identified by any suitable means and at any stage of fish development (e.g. juvenile or adult fish).

According to one embodiment, the loss-of-function mutation in slc45a2 gene is determined phenotypically.

As the loss-of-function mutation in slc45a2 gene is evident by lack of melanin synthesis whereby the fish exhibit an albino-red phenotype, fish comprising the loss-of-function mutation in slc45a2 gene can be identified by their red coloration in the skin and eyes, in the lack of black/grey pigmentation and in the lack of black/grey blotching on the skin (as discussed above).

According to one embodiment, the loss-of-function mutation in slc45a2 gene is determined genotypically.

For example, the loss-of-function mutation in slc45a2 gene can be identified by detecting sequence alterations using, for example, DNA sequencing, next-generation amplicon sequencing (NGS), electrophoresis, an enzyme-based mismatch detection assay and a hybridization assay such as PCR, RT-PCR, High-Resolution Melt curve (HRM), RNase protection, in-situ hybridization, primer extension, Southern blot, Northern Blot and dot blot analysis. Additionally or alternatively, the loss-of-function mutation in slc45a2 gene can also be determined at the protein level using e.g. chromatography, electrophoretic methods, immunodetection assays such as ELISA and western blot analysis and immunohistochemistry.

Moreover, the loss-of-function mutation in slc45a2 gene can be measured or identified at different times during development (developmentally regulated expression or developmental stage-specific expression), in different cell lineages (cell lineage-specific expression), e.g. in somatic or germline cells.

According to one embodiment, the mutation is present in somatic cells.

According to one embodiment, the mutation is present in germline cells.

According to one embodiment, the phenotype is determined prior to the genotype.

According to one embodiment, the genotype is determined prior to the phenotype.

According to one embodiment, the method further comprises breeding the fish.

As used herein the term “breeding” encompasses any reproductive methods that result in heritability of the genetic constitution of a lineage of fish. Such reproductive methods include mating, artificial fertilization, and chromosomal manipulation (such as gynogenesis and androgenesis). Applicable breeding programs include inbreeding, crossbreeding, interspecific or intergeneric breeding and selective breeding.

“Inbreeding” refers to the mating of relatives or fish more closely related than the population average, resulting in inbred offspring. “Crossbreeding” refers to the mating of individuals less closely related than the population average, resulting in hybrid offspring. “Interspecific and intergeneric breeding” refers to the mating of individuals from different species or genera, respectively. “Selective breeding” refers to allowing the superior individuals to mate, based on their phenotypes as compared to control, or based on a known genotype/haplotype.

Different breeding programs can be combined or used in tandem to produce the albino-red tilapia fish having the loss-of-function mutation in slc45a2 gene of the invention.

Any fish hatchery practices and breeding programs known in the art can be used in accordance with the invention. See, for example, Gjedrem, T. 2005, “Selection And Breeding Programs In Aquaculture,” Springer; Tave D, 1999, “Inbreeding and Brood Stock Management,” Fisheries Technical Paper 392, FAO United Nations; Tave D, 1995, “Selective Breeding Programmes,” Fisheries Technical Paper 352, FAO United Nations; Purdom, Colin, 1993, “Genetics and Fish Breeding,” Kluwer; Tave D. 1993, “Genetics for fish hatchery managers,” 2nd ed., Van Nostrand Reinhold, N.Y.; and Kirpichnikov V S, 1981, “Genetic Bases of Fish Selection,” Springer-Verlag, New York; Arai K. “Genetic improvement of finfish species by chromosomal manipulation techniques in Japan,” Aquaculture 197, issues 1-4:205-228, 2001; Khan, T. A., Bhise, M. P. and Lakra, W. S. “Chromosome manipulations in fish—a review.” Indian Journal of Animal Sciences 70: 213-221, 2000; Pandian, T. J. and Koteeswaran, R. “Ploidy induction and sex control in fish,” Hydrobiologia 384: 167-243, 1998.

According to one embodiment, the method of the invention comprises an inbreeding program. Any known inbreeding techniques or programs for producing a new breed or variety can be used. In this method, when a male is determined to be albino-red tilapia fish and having the loss-of-function mutation in slc45a2 gene, that male is bred to many females and a number of his daughters and grand-daughters in order to produce a population of fish that resembles him in phenotype and genotype. For example, a male fish is allowed to mate and its offspring and second generation offspring are allowed to mate with a member of the population; then the male fish is brought back to mate with its great-grand child. Another example of inbreeding involves mating a male individual repeatedly to his daughter, grand-daughter, great-grand daughter, etc. The latter program can produce individuals that are genetically very similar to the male. The resulting inbred offspring can be maintained as a new variety of genetically improved fish. In a specific embodiment, two different inbred lines of fish can be crossbred to produce hybrids with both superior traits.

In another embodiment of the invention, the method comprises a crossbreeding program involving different breeds or varieties (intraspecific crossing), or different species (interspecific crossing), such as fish of different tilapia strains. Crossbreeding increases heterozygosity, and can result in heterosis (or hybrid vigor) wherein the fitness of the offspring exceeds the mean of the average values of the two parental lines. Crossbreeding can involve genetically distant parents, including those of different species or breeds, to develop a new breed with a combination of characteristics of two or more species or breeds. Crossbreeding can be used to increase the viability of a breed by introducing genetic traits for resistance to diseases or changes in environmental factors. Crossbreeding techniques that are well known, such as the techniques used in creating hybrid striped bass, can be applied.

In another embodiment of the invention, the methods comprise a selective breeding program. Selection procedures can operate at the individual level or at the family level, where whole families are selected or culled based on family means (i.e., between-family selection) or where the best fish from each of a number of families are saved (i.e., within-family selection). Fish that are saved become the first generation (F₁) of select brood fish. Their offspring, in turn, are referred to as the “F₂ generation,” etc. The select brood fish is allowed to mate among themselves at random, and this process is then repeated in succeeding generations. Many species exhibit sexual dimorphism in that one sex grows to a larger size or grows faster. If the species does not exhibit sexual dimorphism or if selection will occur before sexual dimorphism begins, then a single cut-off value can be created for the entire population. If the species exhibits sexual dimorphism, separate cut-off values must be created for each sex, or the select population may be composed of only the larger sex.

In individual selection (also known as mass selection), all individuals are assessed, and the decision to select or to cull a fish is based solely on that fish's phenotypic value (e.g. albino-red phenotype).

Family selection differs from individual selection in that the decision to save or to cull fish is conducted at the family level, and individual phenotypic values are important only as they relate to their family's mean. Two types of family selection can be applied: between-family selection and within-family selection, can be used in the methods of the invention. In between-family selection, the mean values for each family are determined, and the mean values are then ranked. Whole families are then either saved or culled. In within-family selection, each family is considered to be a temporary sub-population, and selection occurs independently within each family. When fish are measured to determine which will be saved and which will be culled, the fish in each family are ranked, and the best fish are saved from each family.

According to one embodiment, the method further comprises breeding the fish with a second fish of the tilapia genus to produce a third fish of the tilapia genus having the loss-of-function mutation in the slc45a2 gene.

According to a specific embodiment, fish of the tilapia genus are generated according to some embodiments of the invention, i.e. by introducing into a zygote of the fish a DNA editing agent conferring loss-of-function mutation in the slc45a2 gene. These fish (referred to as F₀) are grown to adulthood and are genetically and phenotypically screened for having the loss-of-function mutation in the slc45a2 gene and for having an albino-red phenotype, respectively. Male fish comprising the loss-of-function mutation in the slc45a2 gene and having an albino-red phenotype (F₀) are crossed with wild-type tilapia female fish (i.e. not having the loss-of-function mutation in the slc45a2 gene). The resultant offsprings (F₁) are selected for having a loss-of-function mutation in one allele of the slc45a2 gene (i.e. being heterozygous for the mutation), and are further mated with a second fish of the tilapia genus having a loss-of-function mutation in one allele of the slc45a2 gene to produce a third fish of the tilapia genus having the loss-of-function mutation in the slc45a2 gene (i.e. being homozygous for the mutation). The resultant offsprings (F₂) are genetically and phenotypically selected for the loss-of-function mutation in the slc45a2 gene (i.e. for being homozygous for the mutation) and for having the albino-red phenotype. The F₂ fish are allowed to mate with a member of the second generation population.

According to another specific embodiment, fish of the tilapia genus are generated according to some embodiments of the invention, i.e. by introducing into a zygote of the fish a DNA editing agent conferring loss-of-function mutation in the slc45a2 gene. These fish (referred to as F₀) are grown to adulthood and are genetically screened for having the loss-of-function mutation in at least one allele of the slc45a2 gene (i.e. for being heterozygous for the mutation). Male and female fish being heterozygous for the loss-of-function mutation in the slc45a2 gene (F₀) are separated and crossed with a second fish being heterozygous for the mutation, to produce a third fish of the tilapia genus having a loss-of-function mutation in the slc45a2 gene (i.e. being homozygous for the mutation). The resultant offsprings (F₁) are genetically and phenotypically selected for having a loss-of-function mutation in the slc45a2 gene and for having the albino-red phenotype, respectively. The F₁ fish are allowed to mate with a member of the first generation population (F₁).

As mentioned, breeding may be carried out by artificial fertilization. This method is specifically warranted in cases in which different species or breeds are crossed. Thus, according to a specific embodiment of the present invention, sperm/eggs can be manually stripped of the fish and fertilized in vitro. Stripping can be carried out using any method known in the art, e.g. by the extraction of ovulated eggs from mature females using a catheter as taught by M. Szczepkowski et al., “A simple method for collecting sturgeon eggs using a catheter”, Arch. Pole. Fich. (2011) 19:123-128, incorporated herein by reference. Accordingly, the eggs or sperm can be removed or sucked off by a vacuum. Additionally or alternatively, the eggs or sperm may be obtained be simply massaging the eggs or sperm out of the abdominal cavity. The fertilized eggs are then treated according to the methods of some embodiments of the invention by introducing into a zygote of the fish a DNA editing agent conferring loss-of-function mutation in the slc45a2 gene. These fish (referred to as F₀) are grown to adulthood and are genetically screened for having the loss-of-function mutation in at least one allele of the slc45a2 gene (i.e. for being heterozygous for the mutation). Male and female fish being heterozygous for the loss-of-function mutation in the slc45a2 gene (F₀) are separated and crossed with a second fish being heterozygous for the mutation, to produce a third fish of the tilapia genus having a loss-of-function mutation in the slc45a2 gene (i.e. being homozygous for the mutation). This crossing may be effected by stripping or by natural mating, as discussed above. The resultant offsprings (F₁) are genetically and phenotypically selected for having a loss-of-function mutation in the slc45a2 gene and for having the albino-red phenotype, respectively. The F₁ fish are stripped again or are allowed to mate with a member of the first generation population (F₁).

As mentioned, the fish being homozygous for the mutation may exhibit the same mutation from both parents. Alternatively, the fish being homozygous for the mutation may exhibit a different mutation in each allele (according to the events in the F₀) and therefore may carry two different mutant alleles.

According to one embodiment, the fish of the tilapia genus are mated with fish of the same strain, i.e. are purebred. Purebred may be an advantage as a genetic source allowing genetic and phenotypic stability.

According to one embodiment, the fish of the tilapia genus are mated with fish of a different strain, i.e. are tilapia hybrids. Hybrids may be an advantage in situations where specific characteristics are warranted (e.g. fish size, temperature stability, salt sensitivity, etc.). Thus, for example, Nile tilapia can be crossed with Mozambique tilapia, Blue tilapia, or Zanzibar tilapia.

According to one embodiment, there is provided a population of fish comprising the fish of some embodiments of the invention, wherein the population is stable for the albino-red phenotype.

The invention also encompasses the fish generated by the methods described, its gametes (sperms and eggs), embryos, and progeny.

As used herein, a progeny of a fish is a fish descended from the first fish by sexual reproduction or cloning, and from which genetic material has been inherited.

According to some embodiments of the invention, the tilapia fish is non-transgenic.

According to some embodiments of the invention, the tilapia fish is transgenic.

According to one embodiment, the tilapia fish is non-genetically modified (non-GMO).

According to one embodiment, the tilapia fish is a genetically modified (GMO).

According to one embodiment, there is provided a feed or food product comprising the fish or part thereof of some embodiment of the invention.

According to one embodiment, the feed or food product being a whole fish, a fish portion, a fish meal or fish oil.

According to a specific embodiment, the feed or food product comprises genomic DNA of the albino-red tilapia according to some embodiments of the invention.

As used herein, “fish meal” refers to meal produced by the boiling of landed fish and other aquatic animal species (either caught or produced), separating out water and oil (e.g. by use of a press), and then drying. Normally fish meal is dried to a moisture content of less than or equal to about 10%, and then the fish meal is distributed at room temperature.

According to a specific embodiment, the fish feed or food product is a surimi, ground fish meat, gelatin, collagen, or fish egg.

According to a specific embodiment, the fish feed or food product is in a solid, a paste or a liquid form.

According to a specific embodiment, the fish feed or food product is fresh, frozen, cooked, boiled, fried or grilled.

The invention also relates to the incorporation the fish products into a non-food or non-feed product, such as e.g. a cosmetic product or a fertilizer.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1 is expressed in a DNA sequence format (e.g., reciting T for thymine), but it can refer to either a DNA sequence that corresponds to an slc45a2 nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Fish Handling

Experiments were approved by the Agricultural Research Organization Committee for Ethics in Using Experimental Animals. Sexually mature Nile tilapia were maintained in 150-L aquaria, in herms consisting of one male and 4-6 females. The temperature was maintained at 24-26° C., and photoperiod of 14L:10D. Herms were constantly monitored for spawning behavior. Fertilized zygotes were collected immediately after natural spawning or by performing in vitro fertilization, as previously described [Fernandes, A. F. A. et al., Reproduction in Domestic Animals (2013) 48: 1049-1055]. When generated by natural mating, larvae for F₁ and F₂ analysis were collected 2-3 days after natural fertilization.

Cloning of Target Sites for gRNAs

Genomic DNA (gDNA) was extracted from fin-clip samples using HotSHOT method as previously described [Meeker, N. D. et al., BioTechniques (2007) 43, 610-614]. Genomic target sequence was downloaded from the UCSC genome browser (www(dot)genome(dot)ucsc(dot)edu/). Amplification of slc45a2-exon1 genomic region (accession no. XM_003451484) was performed using specific primer pairs (Table 1, below) as previously described [Segev-Hadar, A. et al., Frontiers in Endocrinology (2020) 11, 94-94]. Briefly, PCR products were amplified using DreamTaq Green PCR Master Mix (Thermo Fisher Scientific, Vilnius, Lithuania) and analyzed on 1% agarose (LifeGene, Modi'in, Israel) containing Redsafe™ stain (Intron Biotechnology, Seongnam, Korea) in 1× Tris-acetate acid-EDTA buffer (Biological Industries, Kibbutz Beit-Haemek, Israel). PCR products of the predicted amplicon size were extracted from the gel using GEL/PCR Extraction Kit (Hy Laboratories Ltd. Rehovot, Israel), cloned into a pGEM®-T easy vector (Promega, Wisconsin, U.S.A.) and sequenced using T7 primer at Hy Laboratories Ltd. (Rehovot, Israel).

TABLE 1
Oligonucleotides used in this study

| | | Product | |
|---|---|---|---|
| Oligo | 5′-3′ Sequence | size | Use |
| SLC_1F | CTGGCAAACACATCAGCACT (SEQ ID NO: 3) | 500 bp | Cloning |
| SLC_500R | TTCCTCACCCTCACGTTTTC (SEQ ID NO: 4) | | |
| SLC_F_SEQ1 | TGTACAGCTTGGTGTGGTTGAT (SEQ ID NO: 5) | 120 bp | Target |
| SLC_R_SEQ1 | GTGCCAGGATGTAAGGCCTCC (SEQ ID NO: 6) | | analysis |
| sgPRIMER-F | TAATACGACTCACTATAGGGGGAATTCTGCTATGC | 120 bp | gRNA |
| | CGTGGGTTTTAGAGCTAGAAATAGCAAG | | synthesis |
| | (SEQ ID NO: 7) | | |
| sgPRIMER-R | AAAAGCACCGACTCGGTGCCACTTTTTCAAGTTGA | | |
| | TAACGGACTAGCCTTATTTTAACTTGCTATTTCTAG | | |
| | CTCTAAAAC (SEQ ID NO: 8) | | |
| SP1 | ACACTCTTTCCCTACACGACGCTCTTCCGATCTTGT | 185 bp | NGS |
| | ACAGCTTGGTGTGGTTGAT (SEQ ID NO: 49) | | |
| SP2 | GTGACTGGAGTTCAGACGTGTGCTCTTCCGATCTGT | 185 bp | NGS |
| | GCCAGGATGTAAGGCCTCC (SEQ ID NO: 50) | | |

Design of CRISPR Target Sites and Synthesis

sgRNA design and synthesis was performed as previously described [Biran, J. et al., Scientific Reports (2020) 10: 9559] with slight modifications. Briefly, gRNA were designed using CHOPCHOP [Montague, T. G. et al., Nucleic Acids Research (2014) 42: W401-W407], transcribed in vitro using MEGAshortscript™ T7 kit (Life Technologies, United States) and purified using miRNeasy® kit (Qiagen, Germantown, MD, USA). slc45a2 specific crRNAs (i.e. gRNA1-3) and tracrRNA (TRACRRNA05N) were purchased from Sigma-Aldrich Israel Ltd. (Rehovot, Israel) and diluted with 10 mM TRIZMA buffer (Sigma-Aldrich Israel Ltd.). Recombinant-Cas9 (rCas9) protein was produced by the Weizmann Institute of Science Protein Purification Unit (Rehovot, Israel) using the pET-28b-Cas9-His (Alex Schier Lab Plasmids, Addgene, Cambridge, MA, USA) as a template. Sequences of gRNA target sequences and the gRNA used in the study are listed in Tables 2A-B, below.

TABLE 2A
Target sequences of gRNAs used in this study

| Target ID | Target sequence 5′-3′ | PAM | Strand |
|---|---|---|---|
| gRNA1 | AGGGAATTCTGCTATGCCGTGG | TGG | + |
| | (SEQ ID NO: 9) | | |
| gRNA2 | TCGCTGGCTGAACCGATGATGG | TGG | − |
| | (SEQ ID NO: 10) | | |
| gRNA3 | TTGCCGCTCGCCGTGGGGCCGG | CGG | + |
| | (SEQ ID NO: 11) | | |
| sgRNA1 | GGAATTCTGCTATGCCGTGG | AGG | + |
| | (SEQ ID NO: 12) | | |

TABLE 2B
gRNAs used in this study

| gRNA | SEQUENCE |
|---|---|
| gRNA1 | AGGGAAUUCUGCUAUGCCGAUGCUGUUUG |
| | (SEQ ID NO: 13) |
| gRNA2 | UCGCUGGCUGAACCGAUGAAUGCUGUUUG |
| | (SEQ ID NO: 14) |
| gRNA3 | UUGCCGCUCGCCGUGGGGCAUGCUGUUUG |
| | (SEQ ID NO: 15) |
| sgRNA1 | GGAAUUCUGCUAUGCCGUGGGUUUUAGAGCUAGAAAUAGCAAGU |
| (gRNA + constant) | UAAAAUAAGGCUAGUCCGUUAUCAACUUGAAAAAGUGGCACCGA |
| | GUCGGUGCUUUU |
| | (SEQ ID NO: 16) |

Microinjection Procedure

Microinjection to tilapia zygotes and gRNA/Cas9 heterocomplexing were generally performed as previously described [Biran, J. et al., Scientific Reports (2020) 10: 9559]. In short, tilapia zygotes were collected immediately after natural spawning or IVF and chilled on ice to 21° C. to prolong microinjection time at the single-cell stage. Tilapia zygotes at the single-cell stage were injected with a mix containing:
(1) sgRNA1 and rCas9; or
(2) 1-3 of the slc45a2 gRNAs (i.e. gRNA1-gRNA3), tracrRNA and rCas9.

sgRNA/Cas9 or gRNA/tracrRNA/Cas9 mix (100 μmol for each gRNA1-3 and tracrRNA; or 39.72 μmol for sgRNA, mixed with 4.4 μg rCas9 to a final volume of 9 μl) was incubated for 5 min at room temperature to allow the generation of ribonucleoprotein (RNP) heterocomplexes. Microinjection was performed using glass capillaries (1B150E-4 100 mm, WPI, Sarasota, FL, USA) pulled on a Pul-1000 four-step micropipette puller (WPI). Microinjections were carried out using PV 830 Pneumatic Picopump (WPI) calibrated to deliver a volume of approximately 3-5 nL. Following microinjection, zygotes were allowed to develop at 27° C. under constant agitation. Three sets of injection were performed for gRNA1, one set of sgRNA1+gRNA2+gRNA3 and four sets for gRNA2+gRNA3. Each injection set contained approximately 30-50 zygotes with wide mortality range and mutation rate. Control non-injected zygotes were grown separately under the same conditions.

Analysis of Injected Embryos and Offspring

gDNA was extracted from whole embryo 4 days post fertilization (dpf), or fin-clip and sperm from F₀ and F₁ fish, and used for amplification of the target region in the genomic slc45a2-exon1. Amplification products were cloned and sequenced as described above. Retrieved sequences were aligned by MUSCLE (www(dot)ebi(dot)ac(dot)uk/Tools/msa/muscle/).

Next-Generation Amplicon Sequencing (NGS)

The target region of gRNA2 and gRNA3 was amplified from gDNA of 40 injected fish using sp 1 and sp2 primers (Table 1, above). Each amplicon was purified using GEL/PCR Extraction Kit (Hy Laboratories Ltd. Rehovot, Israel). Samples were subsequently used for the generation of sequencing libraries and sequenced according to the Illumina NovaSeq system protocols at Syntezza Bioscience Ltd (Jerusalem, Israel). Data of 39 successfully sequenced samples were analyzed using NGS Cas-analyzer [Park J et al., Bioinformatics (2017) 33: 286-288]. The results were subsequently analyzed manually according to length, gRNA location, indels identified and the reads of each allele. Unmapped sequences were considered insignificant footprint. The frequency of each allele was calculated from total reads.

High-Resolution Melt (HRM) Analysis

gDNA was extracted from fin-clip samples of 330 F₁ fish using HotSHOT method. Each reaction consisted of 5 μL Accumelt™ HRM Supermix (Quanta Biosciences, Gaithersburg, MD, USA), 1 μL DNA (diluted 1:400), 3.4 μl ultra-pure water (UPW), and 0.3 μL of 10 μM for forward and reverse primers each (Table 1, above). The reaction was performed in 96-well plates. Genomic DNA of WT and known mutant alleles were used as control samples and were run in duplicates using a StepOnePlus™ V2.3 Real-Time PCR System (Applied Biosystems, Inc. Foster City, CA, USA). Amplification was performed under the following conditions: 95.0° C. for 10 min, 40 cycles at 95.0° C. for 15 sec, 60.0° C. for 30 sec, 72° C. for 15 sec. Melting curves were generated by a single stage of 95.0° C. for 15 sec, 60.0° C. for 1 min, followed by a temperature increase to 95.0° C. at a 0.3% ramp rate. The database was further analyzed using the Applied Biosystems® High-Resolution Melt Software v3.1 (Thermo Fisher Scientific Inc. CA, USA) with an analysis temperature range of 84.1° C. to 89.9° C.

Melanin Quantification

5-10 scales were randomly sampled from the body centre of 20 randomly selected F₁ fish (5 WT and 15 heterozygous) and were genotyped by HRM. Additionally, scales from F₂ homozygous mutants were sampled to demonstrate a complete melanin loss. Scales were fixed in PFA and then washed 3 times in phosphate-buffered saline. Scales were imaged using Nikon SMZ25 stereoscope. Lateral-line canal positive scales were omitted due to their structural divergence. For each scale, epithelial area was measured and melanophore cell count was performed using ImageJ. Average melanophore density from 2-8 scales/fish was calculated as melanophore/mm 2.

### Example 1

### Identification of the slc45a2 Gene in the Nile Tilapia Genome

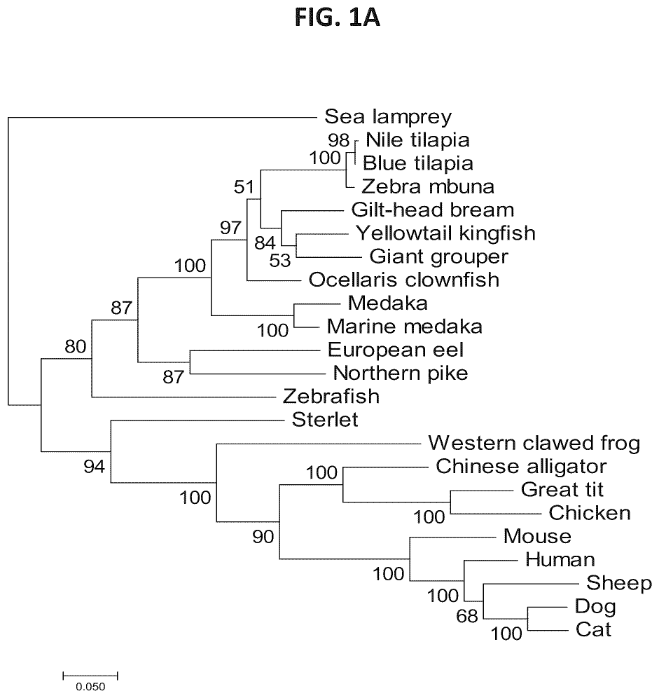

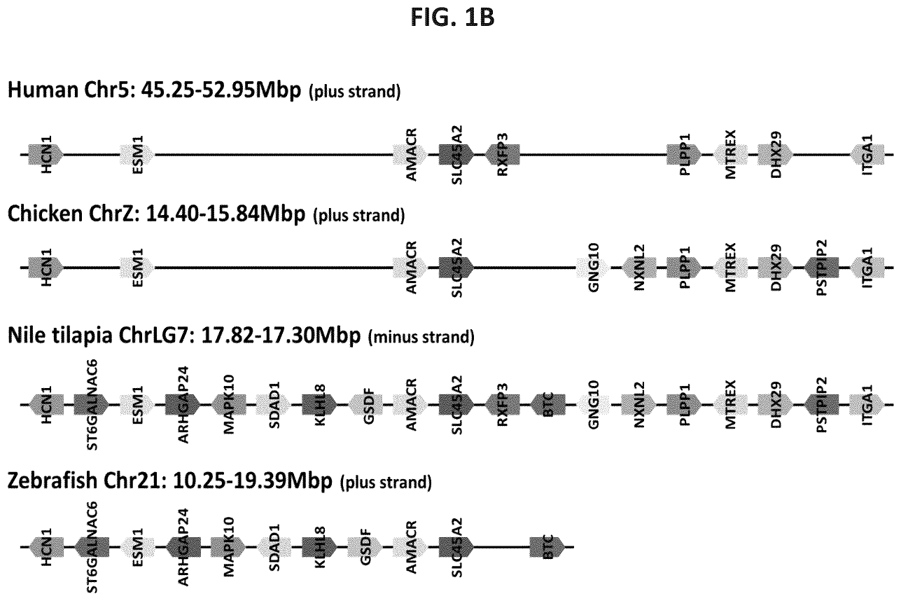

Nile tilapia slc45a2 gene was sought using the previously identified zebrafish slc45a2 mRNA sequence (NM_001110377) [Dooley, C. M. et al., (2013) supra]. Using the zebrafish sequence as the query input, a BLASTn search was performed against the nucleotide collection database of Nile tilapia (taxid: 8128). As expected, this search yielded a single predicted slc45a2 mRNA (XM_003451484) which was localized to chrLG7 (i.e. based on current chromosomal numbering) and shared over 77% identity at the 5′ of the nucleotide sequence. Phylogenetic analysis of slc45a2 mRNA sequences from various vertebrate species including mammals, birds, reptiles, amphibia and fish and using the agnathan sea lamprey as the phylogenetic root demonstrated that slc45a2 is evolutionarily conserved from piscine to human (FIG. 1A). Furthermore, the slc45a2 gene demonstrated high syntenic conservation of its chromosomal region not only with zebrafish, but also with human and chicken (FIG. 1B). These findings support the hypothesis of a common ancestral slc45a2 gene in vertebrates and suggest functional conservation for SLC45A2 throughout evolution.

### Example 2

### Design and Application of CRISPR/Cas9 into Tilapia Zygotes

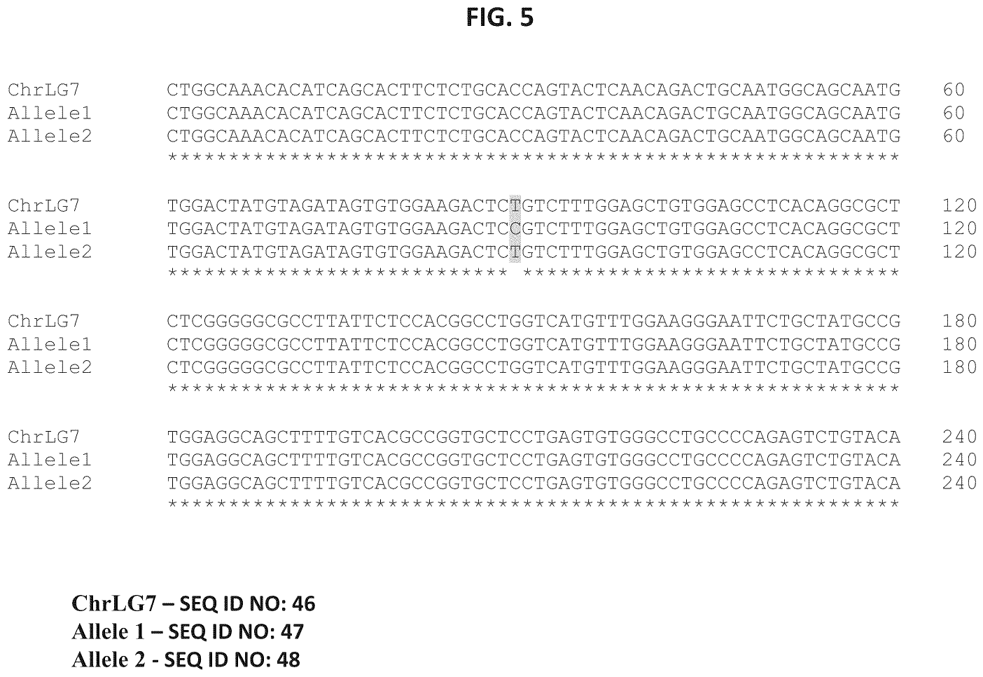

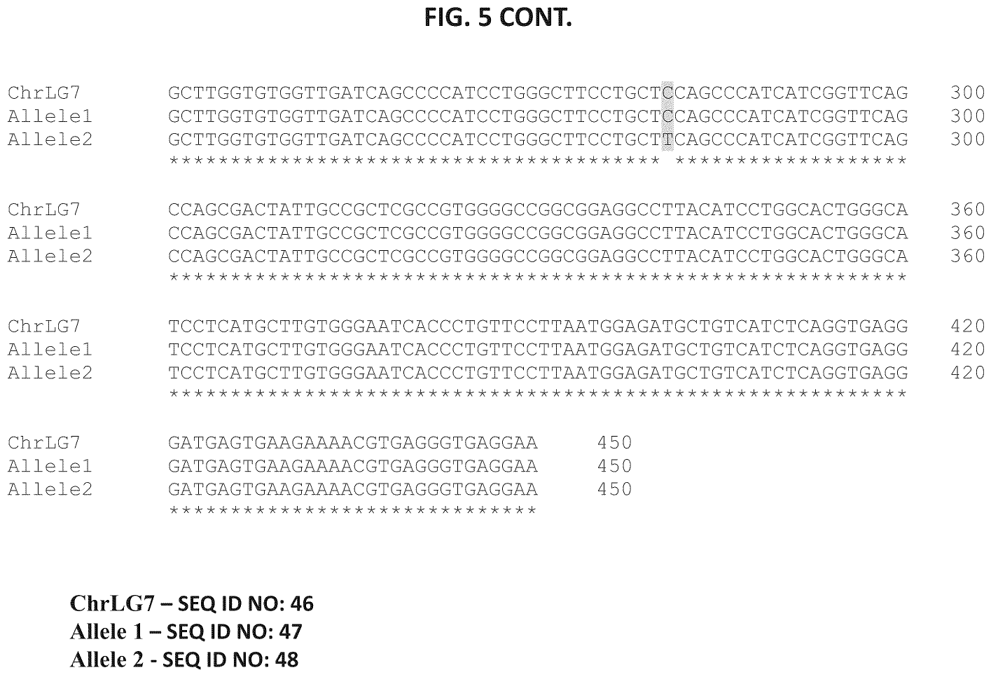

The wide geographical distribution of Nile tilapia results in relatively high genetic diversity. Aiming to design highly specific gRNAs against slc45a2, its exon1 was sequenced in-house on brood stock and two alleles were identified, each differing in one nucleotide from the public sequence of the Nile tilapia genome (FIG. 5). Four specific gRNAs were used, one of which (sgRNA1) was synthesized as single-guide RNA (sgRNA). Before the microinjection, gRNAs were mixed with tracrRNA (trRNA) and recombinant Cas9 (rCas9) protein, to allow the generation of RNP heterocomplex. Similarly sgRNA was mixed with rCas9 protein, to allow the generation of RNP heterocomplex.

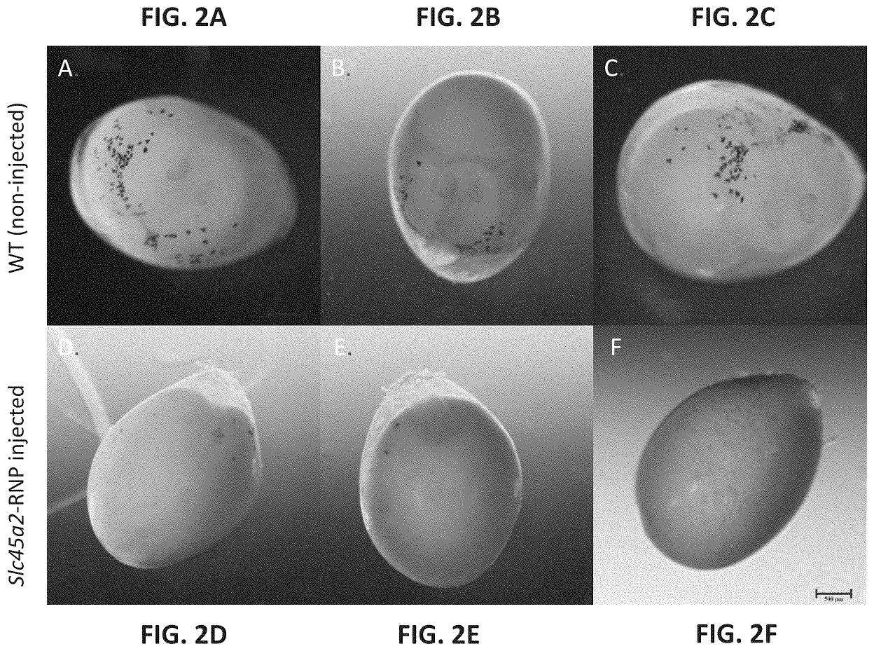

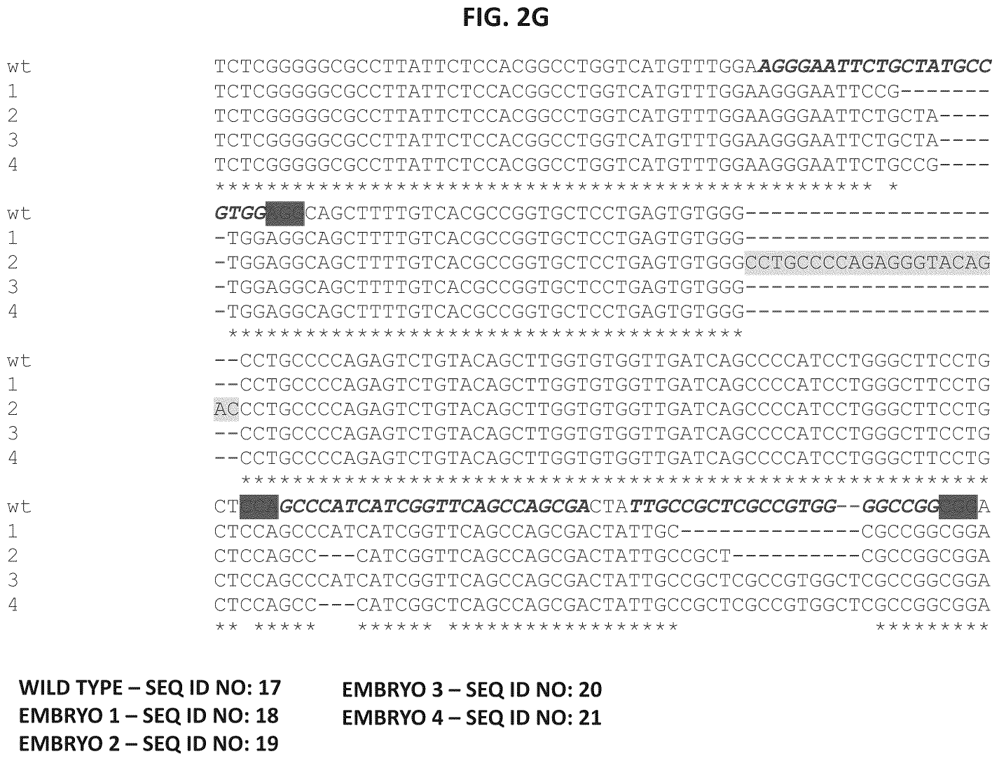

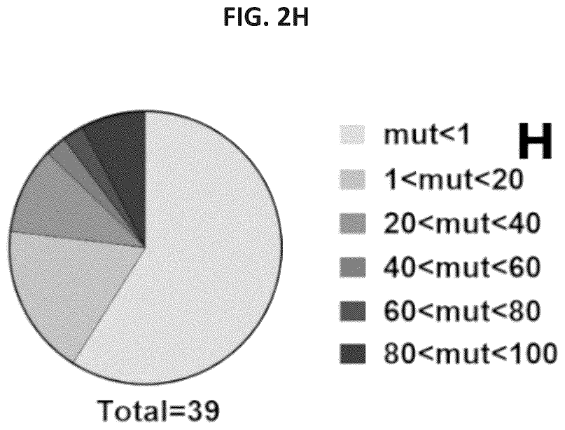

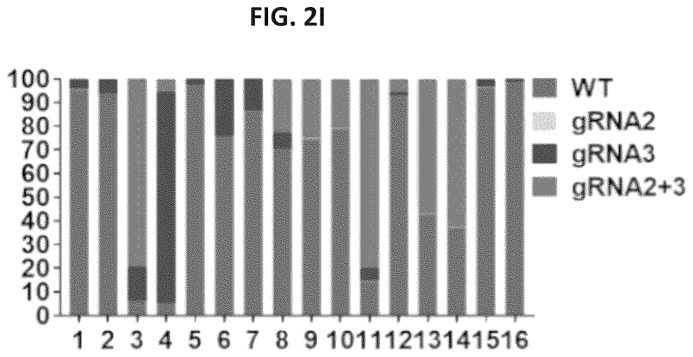

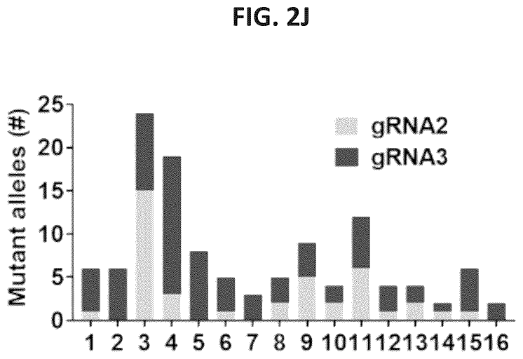

Nile tilapia zygotes at the single-cell stage were microinjected with slc45a2-RNPs in multiplex and the development of melanin was tracked at 4 dpf. At this developmental stage, non-injected tilapia embryos showed prominent amount of melanophores on their body and yolk surface as well as clear melanin formation in the eye (FIGS. 2A-C). Strikingly, larvae injected with slc45a2-RNPs exhibited severe to complete loss of melanin in their body and eyes (FIGS. 2D-F). Analysis of the target regions using gDNA extracted from slc45a2-RNPs injected larvae demonstrated multiple genomic indels, which varied between embryos (FIG. 2G). Furthermore, data showed that both sgRNA and the two-component (gRNA with tracrRNA) could be used to induce genomic indels by microinjection to zygotes.

Aiming to gain higher resolution of the editing events in injected fish, the genomic target region of gRNA2 and gRNA3 from multiplex-injected fish from four different injection sessions was subjected to amplicon sequencing. This analysis revealed variable levels of mutagenic activity in all injected fish (FIG. 2H); however, all fish displayed mutant alleles (data not shown). Analysis of the general mutagenic outcome showed that most mutant alleles resulted from the co-activity of both gRNAs or from sole activity of gRNA3, whereas alleles resulting from exclusive activity of gRNA2 were hardly detected (FIG. 2I). Further analysis of gRNA-specific mutagenic activity showed that the lower activity of gRNA2 was also accompanied by significantly fewer mutagenic outcomes (FIG. 2J). Nonetheless, the combined activity of both gRNAs resulted in the generation of 70 unique alleles (data not shown).

### Example 3

### Generation of Germline with slc45a2 Mutant Alleles

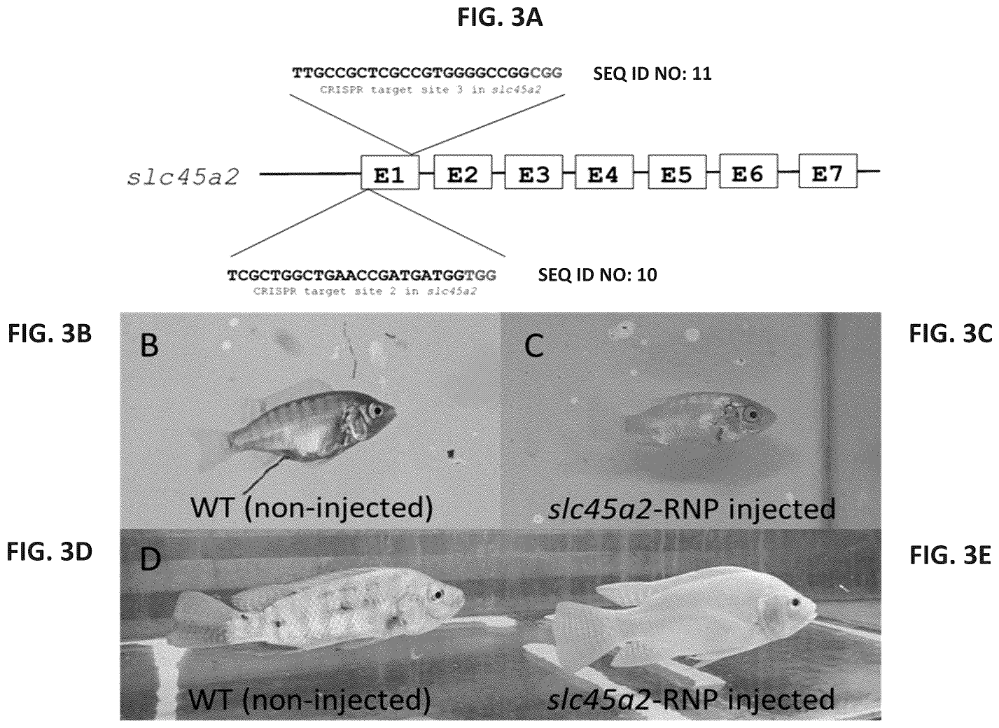

Following the analysis of transient effects of slc45a2 loss-of-function, the present inventors next aimed to induce heritable null mutations in tilapia slc45a2. It was previously demonstrated in zebrafish that multiplexing can increase the mutagenesis throughput and that off-target mutagenesis is of low concern when transiently using the CRISPR-Cas9 in fish zygotes [Varshney, G. K. et al., Genome Research, (2015)]. Hence, slc45a2-exon1 was targeted by co-injection of RNPs containing gRNA2 and gRNA3 (FIG. 3A) into Nile tilapia zygotes. As with triple slc45a2-RNP multiplexing (FIG. 2G) co-injected embryos displayed strong reduction in melanin throughout their body and eyes (FIGS. 3A-E). Specifically, while non-injected siblings exhibited normal pigment development at 1 month post fertilization (FIG. 3B), one of the injected F₀ fish displayed approximately 97-99% loss of melanin in the skin and no melanin was seen in the eyes (FIG. 3C) suggestive of very strong mutagenic activity in its early development. The phenotype of oculocutaneous albinism (OCA), i.e. complete loss of melanin in the eyes and skin resulting in a red phenotype, persisted into sexual maturation of this male (FIG. 3E) as compared to non-injected siblings (FIG. 3D).

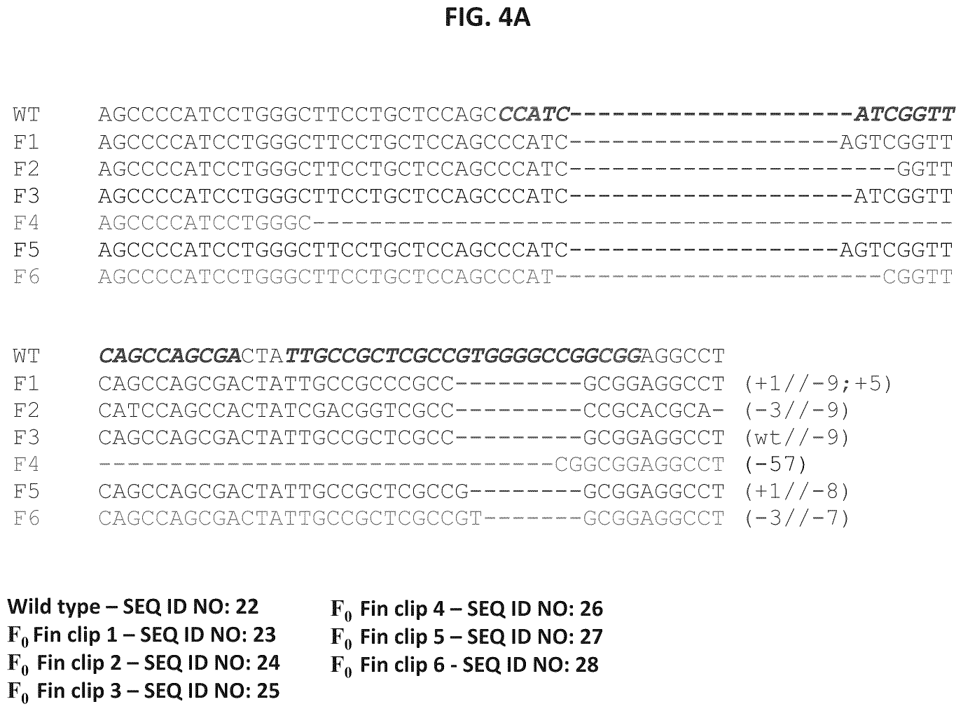

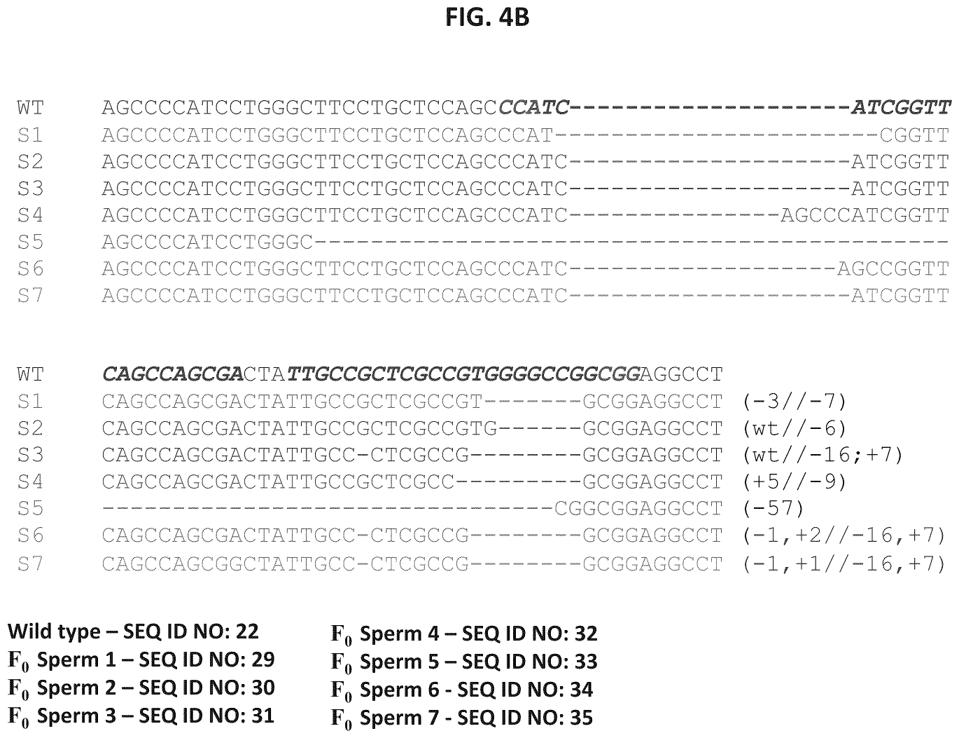

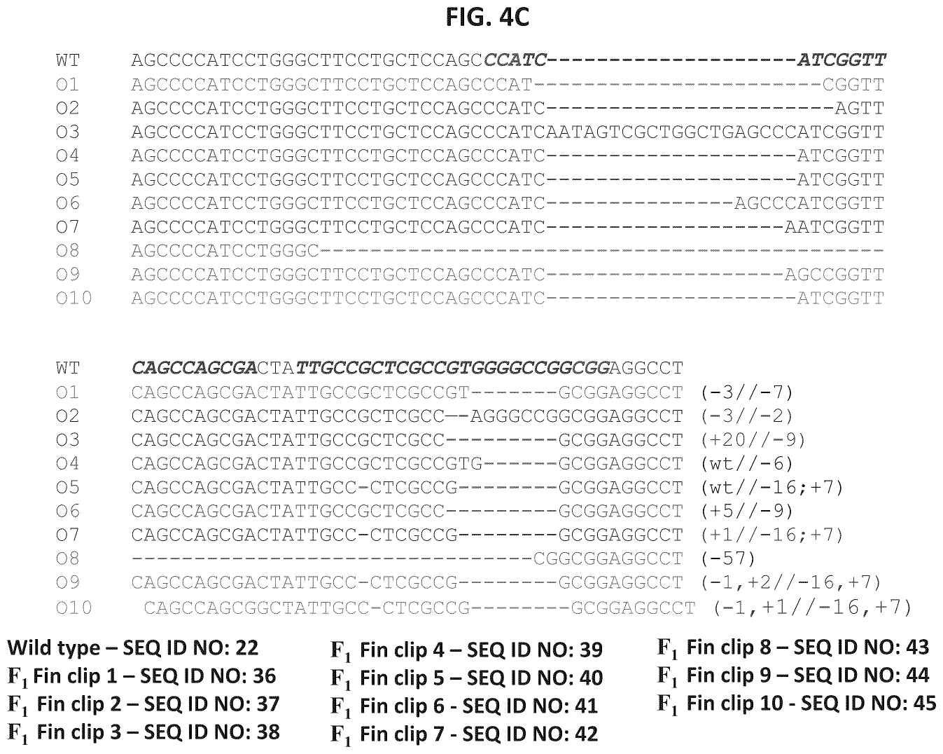

Next, the possible correlation between slc45a2-RNP induced somatic and germline indels was examined. The target region of slc45a2-exon1 was amplified using gDNA extracted from fin-clip and sperm of the albino male and cloned for individual sequencing. Analysis of 10 colonies from fin-clip gDNA and 18 colonies from sperm gDNA identified 6 different indels in the fin and 8 (7 mutant+WT) different products in the sperm, of which 2 were identical between fin and sperm (FIGS. 4A-B) suggesting low-moderate correlation between somatic and germ-cell events. Nonetheless, as sperm was extracted by application of mild physical pressure, some of the identified indles in this sample may have resulted from gDNA that originated from somatic cells of the gonad. Thus, the male was crossed with wild-type (WT) females and sequencing analysis of fin-clips from F₁ fish was performed. This analysis demonstrated that all of the sperm identified mutant alleles as well as the WT allele exist in F₁ heterozygous offspring (FIG. 4C). Taken together, sequencing analysis of gDNA from the F₀ albino mutant and its heterozygous F₁ offspring clearly showed that the novel slc45a2 mutant alleles are heritable and stable.

### Example 4

### High-Resolution Melt (HRM) Analysis and F₂ Phenotyping

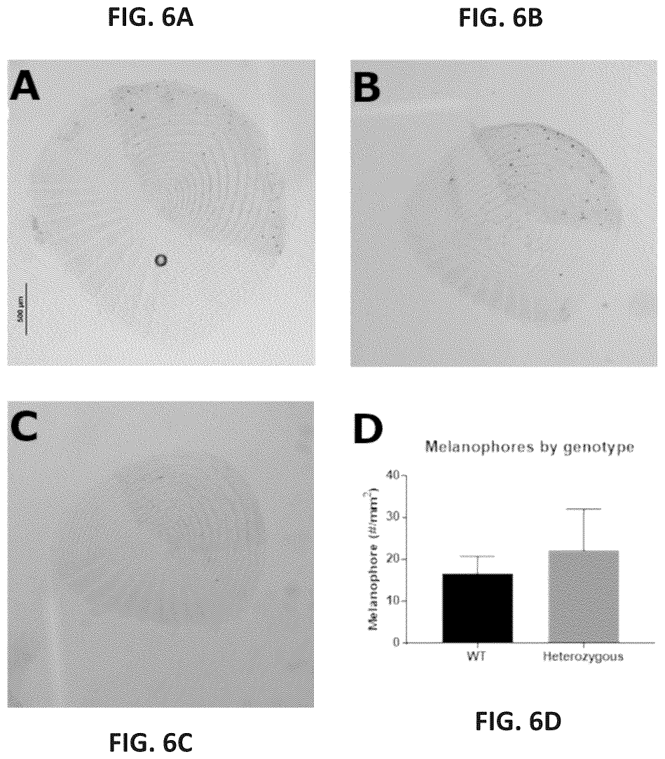

F₁ fish carrying slc45a2^{-/+} genotype displayed no altered pigmentation relative to their WT siblings (FIGS. 6A-D), which raised the need for an efficient method to sort F₁ offspring according to their genotype. HRM was previously shown as an effective method to detect genome editing-driven mutations [Thomas H R et al., PLOS ONE. (2014) 9:e114632]. Due to its low cost, high throughput and high sensitivity, HRM was opted to test its application for the assessment of allele inheritance frequencies in slc45a2^{-/+} F₁ populations. For this, gDNA extracted from tail-clips of 330 F₁ offspring spawned by five random females were analyzed using HRM. The analysis revealed the existence of all mutant alleles that were identified in sperm. Surprisingly, melt curves of some analyzed embryos did not match any of the reference DNAs of known alleles. Sanger sequencing of the target region in these individuals confirmed the existence of three additional mutant alleles within the F₁ population. Thus, ten different heritable mutant alleles were identified (FIG. 4C). These results emphasize the high-throughput and accuracy of this assay for CRISPR-cas9 editing population assessment.

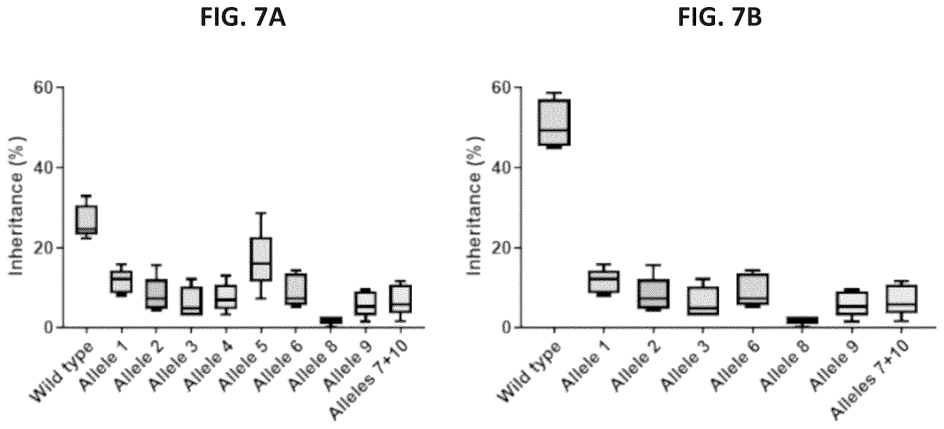

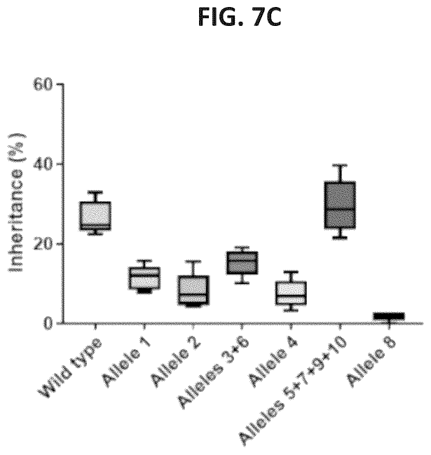

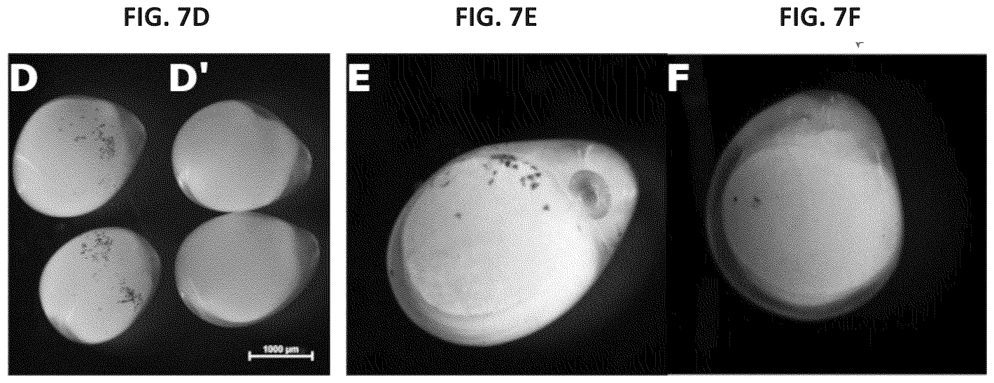

Allele frequencies in F₁ population revealed differential allele heredity (FIG. 7A). 26.7% of the F₁ population were characterize as WT. While allele 05 (single-site indel) displayed the highest inheritance frequency (16.7%), allele 08, which probably resulted from multiplex RNP activity, displayed the lowest allele frequency (1.8%; FIG. 7A). Interestingly, other mutant alleles exhibited frequencies of 5.8%-11.5% in F₁ population with no clear correlation between the genomic CRISPR-Cas9 outcome and its level of inheritance (Table 3, below). Despite the high sensitivity of the HRM analysis, alleles O7 and O10, which differed by two nucleotides, could not be differentiated, therefore, these two alleles were presented together. Analysis of genomic outcomes in each individual site revealed that although both RNPs were microinjected at identical molar ratio, at the level of germline transmission, site 3 showed higher mutagenic response (73% of the F₁ offspring), while site 2 exhibited approximately 50% heritable offspring (FIGS. 7B-C and Table 3, below).

TABLE 3
Allele inheritance frequencies of 330 genotyped F1 offsprings

| | | Allele | Allele | Allele | Allele | Allele | Allele | Allele | Alleles | Allele |
|---|---|---|---|---|---|---|---|---|---|---|
| | Wild type | 1 | 2 | 3 | 4 | 5 | 6 | 8 | 7 + 10 | 9 |
| Batch 1 | 15 | 5 | 3 | 3 | 2 | 18 | 9 | 1 | 1 | 6 |
| Batch 2 | 13 | 7 | 9 | 7 | 4 | 9 | 3 | 0 | 3 | 3 |
| Batch 3 | 17 | 6 | 6 | 6 | 6 | 11 | 4 | 2 | 8 | 3 |
| Batch 4 | 23 | 11 | 5 | 2 | 4 | 5 | 9 | 1 | 4 | 6 |
| Batch 5 | 20 | 9 | 3 | 2 | 9 | 12 | 5 | 2 | 7 | 1 |
| Total (a) | 88 | 38 | 26 | 20 | 25 | 55 | 30 | 6 | 23 | 19 |
| Total (%) | 36.36 | 15.70 | 10.74 | 8.26 | 10.33 | 22.73 | 12.40 | 2.48 | 9.50 | 7.85 |

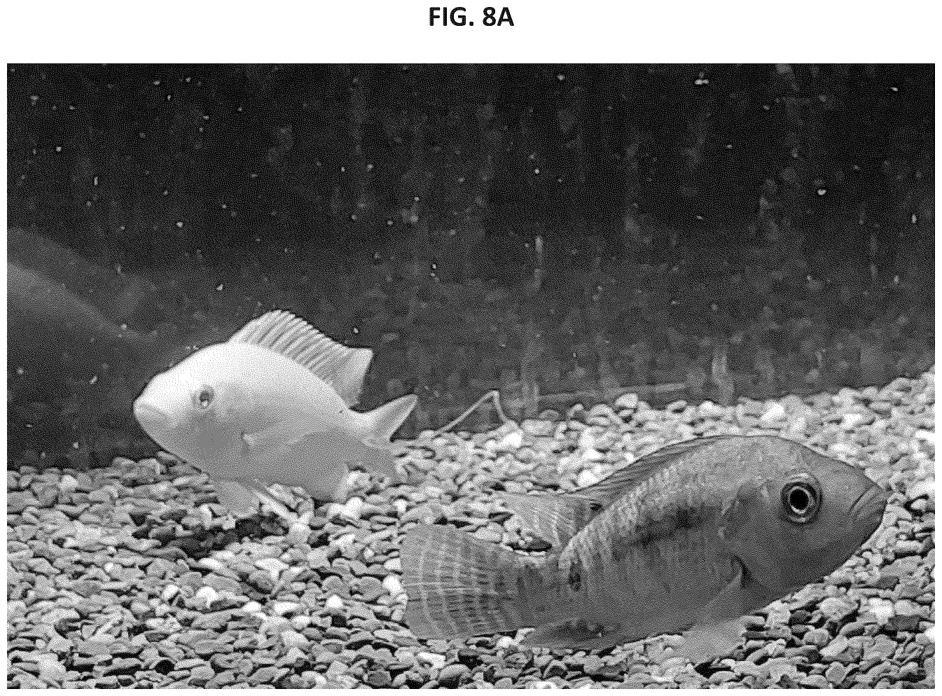

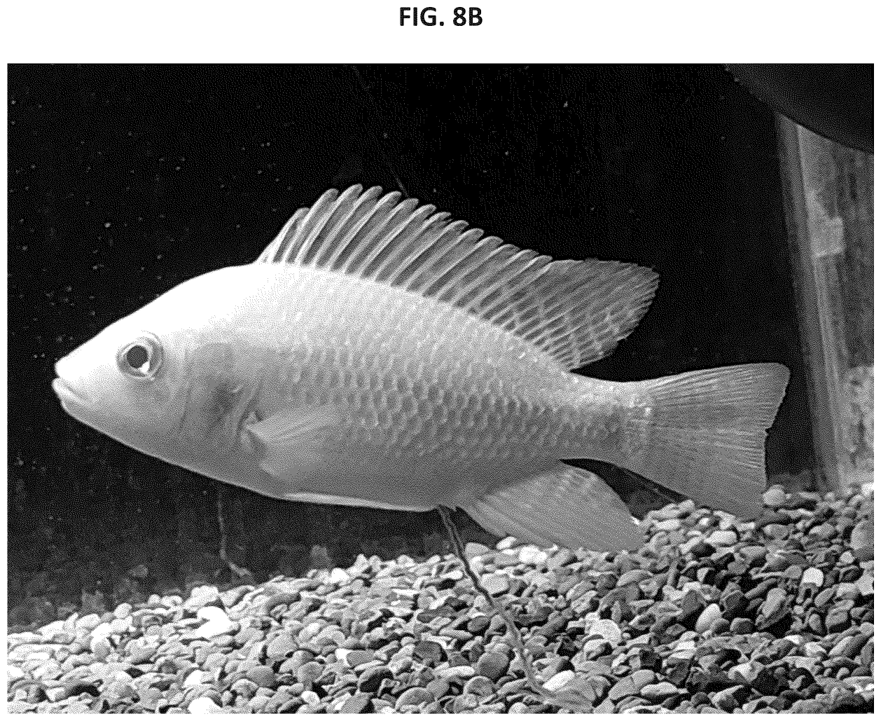

F₂ fish carrying slc45a2^{-/-} genotype were generated by crossing F₁ fish with F₁ fish. Phenotypic analysis of F₂ embryos at 5 dpf showed OCA phenotype with Mendelian inheritance (FIGS. 7D-F). Moreover, this OCA phenotype of F₂ mutants persisted past sexual maturation resulting in albino-red fish with red eyes (FIGS. 8A-B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A fish of a tilapia genus comprising a loss-of-function mutation in a slc45a2 gene, wherein said mutation is in a homozygous form, and wherein said loss-of-function mutation results in an albino-red phenotype of said fish.

2. The fish of claim 1, wherein said albino-red phenotype is devoid of black/grey pigmentation.

3. The fish of claim 1, wherein said albino-red phenotype comprises a red eye phenotype.

4. The fish of claim 1, wherein said loss-of-function mutation is heritable.

5. A method of generating the fish of claim 1, the method comprising:
(a) introducing into a zygote of the fish of the tilapia genus a DNA editing agent conferring a loss-of-function mutation in the slc45a2 gene;
(b) allowing the zygote of step (a) to develop into a fish;
thereby generating the fish.

6. The method of claim 5, further comprising
(c) identifying said fish of said tilapia genus comprising the loss-of-function mutation in the slc45a2 gene.

7. The method of claim 5, further comprising
(d) breeding said fish of step (b) or (c) with a second fish of said tilapia genus to produce a third fish of said tilapia genus having the loss-of-function mutation in the slc45a2 gene.

8. The method of claim 7, wherein said fish and said second fish both carry at least one allele with a loss-of-function mutation in the slc45a2 gene.

9. The method of claim 7, wherein said third fish is homozygous for the loss-of-function mutation in the slc45a2 gene.

10. The fish of claim 1, wherein said mutation is selected from the group consisting of a deletion, an insertion, a point mutation, an indel, and a combination thereof.

11. The fish of claim 1, wherein said mutation comprises two or more mutations in said slc45a2 gene.

12. The fish of claim 1, wherein said mutation is in a target sequence having a sequence selected from SEQ ID NO: 9, 10, 11 and 12 corresponding to said SEQ ID NO: 1.

13. The fish of claim 1, wherein said mutation is expressed in somatic cells.

14. The fish of claim 1, wherein said mutation is expressed in germline cells.

15. The fish of claim 1, wherein said fish of said tilapia genus is purebred.

16. The fish of claim 1, wherein said fish of said tilapia genus is a hybrid.

17. The method of claim 5, wherein said introducing said DNA editing agent comprises introducing two or more DNA editing agents which target distinct sites within the slc45a2 gene.

18. A progeny of the fish of claim 1.

19. A feed or food product comprising the fish of claim 1.
